SEQUENCE LISTING (24 sequences)

SEQ ID NO: 1 - DNA, 19 bp, Artificial Sequence
  synthetic
gaatctctcatcgatctcc

SEQ ID NO: 2 - DNA, 42 bp, Artificial Sequence
  synthetic
agcaaagcataattgtcacctttttggccatggtacatttgg

SEQ ID NO: 3 - DNA, 18 bp, Artificial Sequence
  synthetic
ggcaatcaagccagctat

SEQ ID NO: 4 - DNA, 16 bp, Artificial Sequence
  synthetic
tgtggatcctgctgca

SEQ ID NO: 5 - DNA, 40 bp, Artificial Sequence
  synthetic
gtctcaagggctgttgttctttttgctcagagtcgtcttc

SEQ ID NO: 6 - DNA, 17 bp, Artificial Sequence
  synthetic
gactcctttgagcactg

SEQ ID NO: 7 - DNA, 57 bp, Artificial Sequence
  synthetic
gtgtcaagagctccgagcctcgtcgttcatgcaatagcgctgtggatcctgctgcaa

SEQ ID NO: 8 - DNA, 40 bp, Artificial Sequence
  synthetic
gcgctattgcatgaacgacgaggctcggagctcttgacac

SEQ ID NO: 9 - DNA, 17 bp, Artificial Sequence
  synthetic
atgacgttcgtgttgtt

SEQ ID NO: 10 - DNA, 41 bp, Artificial Sequence
  synthetic
ctccattctggttactgccatttttatcagcgaaatgcacc

SEQ ID NO: 11 - DNA, 18 bp, Artificial Sequence
  synthetic
tctgagggtccaccaaac

SEQ ID NO: 12 - DNA, 19 bp, Artificial Sequence
  synthetic
atactgcgtcttggttcac

SEQ ID NO: 13 - DNA, 39 bp, Artificial Sequence
  synthetic
cggccccaaggtttacctttttccatgttgagtgagagc

SEQ ID NO: 14 - DNA, 17 bp, Artificial Sequence
  synthetic
ggtgttaattggaacgc

SEQ ID NO: 15 - DNA, 58 bp, Artificial Sequence
  synthetic
gtgtcaagagctccgagcctcgtcgttcatgcaatagcgctctgagggtccaccaaac

SEQ ID NO: 16 - DNA, 40 bp, Artificial Sequence; identical to SEQ ID NO: 8
  synthetic

SEQ ID NO: 17 - DNA, 18 bp, Artificial Sequence
  synthetic
ggagagtgagttgatcag

SEQ ID NO: 18 - DNA, 41 bp, Artificial Sequence
  synthetic
atagccctcctaggctcctttttccctctatctgcaacttg

SEQ ID NO: 19 - DNA, 18 bp, Artificial Sequence
  synthetic
aggcttgcttacctccag

SEQ ID NO: 20 - DNA, 19 bp, Artificial Sequence
  synthetic
cagaggcacctaggattgg

SEQ ID NO: 21 - DNA, 39 bp, Artificial Sequence
  synthetic
tggtgacctgaactagggtttttgtgctgtgatctgtcc

SEQ ID NO: 22 - DNA, 18 bp, Artificial Sequence
  synthetic
ctttccctcatccttctc

SEQ ID NO: 23 - DNA, 59 bp, Artificial Sequence
  synthetic
gcagcggacgtcatagggacaatatctttctcgcgcgggacagaggcacctaggattgg

SEQ ID NO: 24 - DNA, 40 bp, Artificial Sequence
  synthetic
tcccgcgcgagaaagatattgtccctatgacgtccgctgc

(19) United States
(12) Patent Application Publication
(10) Pub. No.: US 2021/0395810 A1
(43) Pub. Date: Dec. 23, 2021

(54) Processes to Detect Coronaviruses

(71) Applicants: Steven A BENNER, Gainesville, FL (US); Ozlem Yaren, Gainesville, FL (US)

(72) Inventors: Steven A BENNER, Gainesville, FL (US); Ozlem Yaren, Gainesville, FL (US)

(21) Appl. No.: 16/996,154

(22) Filed: Aug. 18, 2020

Related U.S. Application Data
(63) Continuation-in-part of application No. 15/826,126, filed on Nov. 29, 2017, now Pat. No. 10,920,267.
Application No. 15/826,126 is a continuation-in-part of application No. 16/168,349, filed on Oct. 23, 2018, now abandoned.
Application No. 16/168,349 is a continuation-in-part of application No. 15/826,126, filed on Nov. 29, 2017, now Pat. No. 10,920,267.
(60) Provisional application No. 62/427,868, filed on Nov. 30, 2016.

(51) Int. Cl. C12Q 1/6853; C12Q 1/6876

(52) U.S. Cl. CPC C12Q 1/6853; C12Q 1/6876; C12Q 2565/60; C12Q 2521/107; C12Q 2563/107

(57) ABSTRACT

Processes and devices are disclosed that detect RNA and DNA in a sample, including without limitation RNA from coronaviruses, with said processes generating a fluorescent signal if a segment of a specific RNA molecule is present in a sample.

### CROSS REFERENCE TO RELATED APPLICATIONS

This application claims priority to U.S. patent application Ser. No. 15/826,126, filed 29 Nov. 2017, for “Isothermal amplification of oligonucleotides”, which is co-pending and claimed U.S. Provisional Application No. 62/427,868 (filed 30 Nov. 2016). This application also claims priority to U.S. patent application Ser. No. 16/168,349, filed 23 Oct. 2018, for “Processes for Point of Care Detection of DNA and RNA”, which is co-pending and also claimed in part U.S. patent application Ser. No. 15/826,126, filed 29 Nov. 2017.

### STATEMENT OF RIGHTS TO INVENTIONS MADE UNDER FEDERALLY-SPONSORED RESEARCH

Not applicable.

### THE NAMES OF PARTIES TO A JOINT RESEARCH AGREEMENT

Not Applicable

### INCORPORATION BY REFERENCE OF MATERIAL SUBMITTED ON A COMPACT DISK

None

### BACKGROUND OF THE INVENTION

### 1. Field of the Invention

This invention relates to processes for detecting DNA and RNA molecules, especially those that arise from infectious diseases, in particular RNA viruses. More specifically, it provides specific conditions that allow the detection in less than 30 minutes in unprocessed samples. Still more specifically, it concerns devices that can be used outside of clinical laboratories, including hand-held devices to be used by individuals in, and processes to be used in kiosks at entrances to public spaces and workplaces.

### 2. Description of the Related Art

Methods that detect small numbers of nucleic acid molecules (which include DNA molecules and RNA molecules, collectively “xNA” molecules) from pathogens and other biological agents are useful in diagnostics, research, and biotechnology. In general, the number of xNA molecules that a method must detect to be useful are too few for them to be detected directly. Accordingly, detection methods often begin with an amplification step.

Amplification means a process that yields many product xNA molecules, where the production of those molecules requires a starting xNA sequence, a “target” or an “analyte”. Generally, the product xNA molecules (“amplicons”) contain within them one or more segments of DNA whose sequence corresponds to the sequence of a part of the target xNA molecule, or its Watson-Crick complement. These segments arise by polymerase-catalyzed copying of the xNA molecule. However, useful amplification methods often incorporate additional segments into the amplicons, whose sequences arise from tags on primers.

Classically, amplification has been done using the polymerase chain reaction (PCR) [R. K. Saiki, D. H. Gelfand, S. Stoffel, S. J. Scharf, R. Higuchi, G. T. Horn, K. B. Mullis, H. A. Erlich (1988) Primer-directed enzymatic amplification of DNA with a thermostable DNA polymerase. Science 239, 487-491]. Here, a “forward primer” that is substantially Watson-Crick complementary to a pre-selected region of a DNA target is annealed to the target to form a duplex. Next, the primer-target complex is incubated with a DNA polymerase and the appropriate 2′-deoxynucleoside triphosphates to yield a Watson-Crick complementary DNA molecule; the target and its complement, as it is formed, are bound in a double stranded double helix. The double strand is then “melted” by heating, typically to temperatures above 75° C., to give the two complementary DNA strands in single stranded form. The mixture is then cooled so that the original target binds to a second forward primer, while its complement binds to a “reverse primer”, which is designed to bind to a preselected segment downstream in the product DNA molecule. Then, polymerase extension is repeated, with both primers extended to give full-length products, again as duplexes (now two in number). The results are multiple copies of a segment of the target molecules between the primer binding sites, as well as multiple copies of the complement. In asymmetric PCR, the ratio of these two primers is different from unity. Non-target sequences can be added to the amplicons from tags on the 5′-ends of those primers.

Temperature cycling to separate the two strands in PCR is undesirable in many applications, including applications that amplify target xNA, for its detection, at entrances to public spaces. Thus, the art contains many methods that seek amplification methods that do not need temperature cycling. These include “recombinase polymerase amplification” (RPA) [Piepenburg, O., Williams, C. H., Stemple, D. L., Armes, N. A. (2006) DNA Detection using recombination proteins. PLoS Biol 4 (7): e204], rolling circle amplification (RCA), NASBA, helicase-dependent amplification (HDA) [Tong, Y., Lemieux, B., Kong, H. (2011) Multiple strategies to improve sensitivity, speed and robustness of isothermal nucleic acid amplification for rapid pathogen detection. BMC Biotechnol. 11 Art. No: 50][Lemieux, B., Li, Y., Kong, H. M., Tang, Y. W. (2012) Near instrument-free, simple molecular device for rapid detection of herpes simplex viruses: Expert Review Molec. Diagnostics 12, 437-443 DOI: 10.1586/ERM.12.34] and LAMP, among others. These are called “isothermal amplification” methods.

Isothermal amplification methods frequently do not perform well, however. In many cases, the extent of amplification appears to depend on the specific sequence being amplified or (perhaps) the sequence of probes and/or primers used in the amplification. In some cases, amplification fails entirely. In other cases, extra “spurious” products are observed in addition to the target amplicon. Spurious products are especially often seen when isothermal amplification is attempted for more than one target nucleic acid in a single sample (“multiplexing).

Theory is generally unable to predict variable results, although speculation can be found in the art, sometimes informal and sometimes contradictory. Without being exhaustive, speculative suggestions include the possibility that at low temperatures, non-Watson Crick interactions might cause some of the DNA molecules involved (primer, probe, or analyte) to fold in a way that defeats the amplification process. Others have suggested that high temperatures must be regularly traversed to avoid an (often unknown) intra- or intermolecular interaction from capturing the system as an artifact. Primer-primer interactions have been invoked to explain failure of various isothermal amplification systems, especially when is multiplexing is attempted.

One isothermal amplification method is called “loop-mediated isothermal amplification” (LAMP) [Kubota et al. (2013) Patent Application Publication (10) Pub. No.: US 2013/0171643 A1 Kubota et al. (43) Pub. Date: Jul. 4, 2013 (54) Sequence Specific Real-Time Monitoring Of Loop-Mediated Isothermal Amplification (LAMP)]. The LAMP process comprises a reaction involving one or more LAMP primers that bind in a Watson-Crick sense to the target xNA. As illustrated in FIG. 1, LAMP may employ six primers that bind by Watson-Crick complementarity to eight distinct regions within the target analyte. The primers for LAMP are designated as internal primers (FIP and BIP), outer primers (F3 and B3), and loop primers (LB and LF).

LAMP is initiated by adding internal primers (FIP or BIP) that annealed by Watson-Crick complementarity to regions (F2c or B2c) within the target xNA analyte. The outer primer (F3 or B3) then hybridizes to its priming site (F3c or B3c) on the target xNA and initiates the formation of self-hybridizing loop structures by strand invasion of the DNA sequences already extended from the internal primers (FIP and BIP). The resulting dumbbell structure then becomes a seed for exponential LAMP amplification by a strand displacing polymerase.

The synthesis of product molecules process is further accelerated by the loop primers (LF and LB), which are designed to hybridize in oligonucleotide segments between F1c and F2; these are called B1c and B2, respectively, in FIG. 1.

LAMP reactions are generally run under isothermal conditions. Temperatures are commonly fixed at a value between 60° C. and 70° C., sometimes marginally lower, sometimes marginally higher. The amplicons are concatemers of the region in the target that is targeted, and may fold to form “cauliflower-like structures” with multiple loops. The dumbbell structures then are seeds for further amplification.

One challenge of the LAMP process is the visualization of the products that are formed. Classically, the progress of LAMP may be followed by measuring the turbidity in the reaction mixture arising from precipitating magnesium pyrophosphate, a by-product of LAMP reaction. In real-time analysis, the creation of LAMP products may be monitored by adding intercalating dyes to the mixture. Such dyes include SYBR Green® or EvaGreen®. When double-stranded DNA is formed, these dyes bind and, once bound, fluoresce.

These processes do not allow the sequence of the DNA product to be confirmed. Thus, the formation of other products unrelated to the target can give a false positive signal.

Alternative approaches for detecting the products of LAMP-type amplification can be specific for the target sequence. These include the use of molecular beacons [Yaren, O., Bradley, K. M., Moussatche, P., Hoshika, S., Yang, Z., Zhu, S., Karst, S. M., Benner, S. A. (2016) A norovirus detection architecture based on isothermal amplification and expanded genetic systems. J. Virol. Methods 237, 64-71], which is incorporated herein in its entirety by reference. Here, a molecular beacon comprises a Watson-Crick self-complementary stem and loop structure that is conjugated to a fluorescent molecule at one end and a quencher molecule at the opposite end. The loop sequence is Watson-Crick complementary to an analyte. In the absence of the analyte, no fluorescence is seen, as the fluorophore and the quencher remain in close proximity. When the loop region hybridizes to the target, however, the quencher and fluorophore are separated from each other, and the beacon emits light via fluorescent emission. However, the use of molecular beacons for real-time monitoring LAMP can be difficult, since stem structure may not be stable at the temperature where LAMP is run. Nevertheless, it may be a useful technique for end-point detection of LAMP amplicons.

An alternative way of visualizing the products uses an “assimilating probe” [Kubota, K., Jenkins, D. M., Alvarez, A. M., Su, W. W. (2011) Fret-based assimilating probe for sequence-specific real-time monitoring of loop-mediated isothermal amplification (LAMP). Biol. Eng. Trans. 4, 81-100]. This adds two more components to the LAMP reaction mixture, specifically, two DNA strands that hybridize over part of their segment by Watson-Crick complementarity. The first oligonucleotide strand has a fluorescence quenching moiety covalently attached at its 3′ end; the second DNA strand of the assimilating probe has a fluorophore covalently attached at its 5′-end. When the two strands are hybridized, the quencher and fluorophore are brought into close proximity, and no fluorescence is seen.

To work, this “assimilating probe” must also have a single stranded region attached to the 3′-end of the fluorescently tagged oligonucleotide. This is a priming sequence that is substantially complementary to a selected segment of the target analyte xNA. The second oligonucleotide strand and the first oligonucleotide strand added to the LAMP reaction are preferably in a ratio of 1:1, although Kubota teach that the ratio in the mixture may be less than 1:1. The art teaches a preferred concentration of the assimilating probes between about 0 μM to about 1 μM.

In assimilating probe LAMP, the priming region of the fluorescently tagged oligonucleotide is extended by a strand-displacing DNA polymerase or reverse transcriptase, with the target analyte xNA being used as a template for the extension. During the LAMP, primer extension from reverse primers then reads through the primer on the fluorescently tagged oligonucleotide, and then the segment of DNA from the fluorescently tagged oligonucleotide itself. This read through displaces the oligonucleotide that bears the quencher. This separates the florescent species from the quenching species, allowing the fluorescence to be observed and measured from the fluorescently tagged oligonucleotide that has been “assimilated” into the LAMP products.

The process taught by Kubota (2011) for visualizing the products of LAMP suffers from various limitations. First, the LAMP amplification product mixture is what becomes fluorescent. However, LAMP does not produce a single product. Rather, it produces a series of product concatemers. This means that the fluorescence is not present in a single molecule that can be captured and observed directly, but rather is distributed among multiple molecules that behave differently, not only on gel electrophoresis, but also by any other separation method.

Further, as taught in Kubota (2011), the two strands in the assimilating probe are held together by Watson-Crick pairing between standard nucleotides. As natural biological samples contain many xNA molecules built from natural nucleotides, these can invade the duplex of the assimilating probe, separate fluorophore and quencher even in the absence of LAMP, creating false positives.

Further, especially when LAMP is multiplexed, the multiple strands of nucleic acid that are added can interact with each other in the presence of polymerases to form undesired products, including primer dimers. These can consume LAMP resources unproductively.

A displaceable architecture that releases a fluorescently tagged species was reported for primers that carried a quencher by Tanner et al. [Tanner, N. A., Zhang Y Fau—Evans, T. C., Jr. and Evans, T. C., Jr. (2012) Simultaneous multiple target detection in real-time loop-mediated isothermal amplification. BioTechniques 53, 81-89]. However, these prime internally to the loop, regions Tanner does not teach a process where a fluorophore-releasing probe primes by Watson-Crick complementarity into the loop regions.

Further, neither Tanner (2012) nor Kubota (2012) teach the use of nonstandard nucleotides in the tag regions that hold together the fluorescently labeled oligonucleotide and the quencher oligonucleotide. Here, “nonstandard nucleotides refers to nucleotides built from an artificially expanded genetic information system” (AEGIS) (FIG. 12). AEGIS components have nucleobase analogs with their hydrogen bonding groups are shuffled. This creates new orthogonally binding nucleobase pairs, which cannot hybridize to any natural nucleotide present in any biological sample. Neither Tanner (2012) nor Kubota (2011) teach the use of nonstandard nucleotides in the regions of the primers that bind to the targets or any of the product loop regions.

Finally, neither Tanner (2012) nor Kubota (2012) teach the use of nonstandard nucleotides in the primer regions made from self-avoiding molecular recognition systems (SAMRS FIG. 13) [Hoshika, S., Leal, N., Chen, F., Benner, S. A. (2010) Artificial genetic systems. Self-avoiding DNA in PCR and multiplexed PCR. Angew. Chem. Int. Edit. 49, 5554-5557.]. These prevent primers, present in many types in a standard LAMP architecture, and present in multiplicatively higher numbers in multiplexed detection systems, from interacting with each other.

A parent application of the instant application offered an invention that changed the architecture of the process by placing the fluorescent species on the displaced oligonucleotide, and the quencher on the priming oligonucleotide, and the primer at the 3′-end of the displaceable probe priming on the loop region of an amplifiable structure, rather than on the target analyte itself. This allows the fluorescent species to be a single molecule whose sequence is unrelated to the sequence of the target analyte, and to be released only after the amplification fully starts. This, in turn allows it to be captured, even while the amplification is occurring. This signal sequence is also not spread over many amplicons.

Further in this “displaceable probe LAMP” (DP-LAMP), the two components of the reverse displaceable probe may optionally hybridize via pairing with nonstandard nucleotides AEGIS. The advantages of this are several. AEGIS:AEGIS pairing prevents invasion of the displaceable probe by natural nucleic acids, preventing false positives in complex biological mixtures. Further, this allows the displaced fluorescent probe to be captured in real time, even as the amplification is taking place

Further in the instant invention, self-avoiding molecular recognition nucleotides may be placed in the priming oligonucleotides. This prevents the primers from interacting with each other to produce artifacts and wasting amplification resources.

Further in DP-LAMP, isothermal amplification can be initiated by a target oligonucleotide that is adsorbed on a solid phase containing quaternary ammonium groups. This is called “Q-paper”, and is covered by U.S. patent application Ser. No. 16/168,349, whose content is entirely incorporated by reference in this disclosure. Also incorporated herein in their entirety by reference are: Yaren, O., Bradley, K. M., Moussatche, P., Hoshika, S., Shu, Z., Karst, S. M., Benner, S. A. (2016) A norovirus detection architecture based on isothermal amplification and expanded genetic systems. J. Virol. Methods 237, 64-71.

Yaren, O., Glushakova, L. G., Bradley, K. M., Hoshika, S., Benner, S. A. (2016) Standard and AEGIS nicking molecular beacons detect amplicons from the Middle East Respiratory Syndrome coronavirus, J. Virol. Methods 236, 54-61.

Yaren, O., Alto, B. W., Bradley, K. M., Yang, Z., Benner, S. A. (2017) Point of sampling detection of Zika virus within a multiplexed kit capable of detecting dengue and chikungunya. BMC Infect. Diseases 17.1, 293

The demands placed on xNA detection are especially severe with pathogen such as 2019-nCoV coronavirus. 2019-nCoV has a (i) its stunning range of symptoms, from death to none at all, (ii) the very high viral load in some infected individuals, and (iii) the ability of asymptomatic individuals to infect others, including that ability among those who never develop symptoms. This places a high premium on tests that can be run in between 15 and 30 minutes at the entrances to public space, returning test results essentially immediately, and allowing decisions to be made in real time about whom to send out for a standard test, and who can enter the public space without fear of forward contamination.

### BRIEF SUMMARY OF THE INVENTION

This invention offers processes to detect xNA with specifications that meet these requirements:

(a) The sample need not leave a building where an individual gives it.

(b) Does not involve any sample preparation other than collection, transfer and dilution.

(c) Has low cost disposables.

(d) Takes less than 30 minutes to deliver an easily read signal.

(e) Has limits of detection (LODs) adequate to detect risk of forward infections.

(f) Uses directly mid-turbinate nasal swabs and saliva.

Further, this invention covers hand-held devices that allow the signal to be generated and observed by a user, a “personal” RNA virus detection device that is made possible because of steps (a) through (f). The claimed devices comprise (i) a microprocessor that controls (ii) a heater that warms to between 50 and 70° C. (iii) a disposable that contains reagents that generated a fluorescent signal when viral RNA is present, together with (iv) a light that illuminates the region of the disposable that generates the fluorescent signal, and (v) a port that allows the user to visualize the fluorescent signal.

### BRIEF DESCRIPTION OF THE DRAWINGS

FIG. 1A. Displaceable probe DP-RT-LAMP architecture, real-time analysis, observed during the amplification, and end-point visualization, occurring at the end of the amplification. DP-RT-LAMP is initiated by adding primers (FIP or BIP) that anneal to F2c or B2c regions. Outer primer (F3 or B3) then hybridizes to F3c or B3c and initiates formation of self-hybridizing loop by strand invasion of the DNA sequences already extended from the internal primers (FIP and BIP). The resulting dumbbell structure is then used as a seed for exponential LAMP amplification by a strand displacing polymerase. This process is further accelerated by the introduction of loop primers (LF or LB) hybridizing to segments between F1c and F2 or B1c and B2, respectively. Further, priming region of the quencher labeled probe (e.g. LB) is extended by a strand-displacing polymerase, and primer extension from the reverse primers then reads through the primer on the quencher labeled probe, displacing the probe that bears the fluorophore. This architecture differs from standard LAMP in how the signaling element moves in the detection architecture.

FIG. 1B. Increase in the fluorescent signal and real-time analysis of the process manifests itself as sigmoidal curve as it would in RT-qPCR using TaqMan probes.

FIG. 1C. In addition to real-time analysis, end-point fluorescence can be visualized using an observation box with blue LED exciting at 470 nm through orange filter (Firebird Biomolecular Sciences LLC, US).

FIG. 2. Limits of detection (LODs) determined for the assay using RNA target obtained by in vitro transcription of a synthetic DNA molecule corresponding to a region of the 2019-nCoV genome, and using the CoV2-W3 primer set. Incubation at 65° C. only, without pre-incubation at 55° C. Two traces with no-template control (NTC) superimpose, and give no signal. The 500, 50, 25, and 5 copies give progressively longer times to pass a threshold (Tt). This proves the sensitivity of the test in fully contrived samples.

FIG. 3. Limits of detection (LODs) determined for the assay using full length RNA target (Twist) and the CoV2-W3 LAMP primer set. Assays were run at 65° C. for 60 min using the LightCycler® 480. The determined LOD was 100 copies/assay with threshold time (Tt) of 25.31 min. Traces with no-template control (NTC), one copy, and 10 copies superimpose, and give no signal.

FIG. 4. Limits of detection (LODs) determined for the assay using whole viral SARS-CoV-2 that was heat-inactivated (65° C., 30 min, from BEI Resources). Different enzymes and buffers systems were tested as well as incubation temperature was modified. The determined LOD was 10 copies/assay with a Tt of 16 min using Condition 1.

FIG. 5A-FIG. E. Limit of detection on LAMP primers using heat-inactivated SARS-COV-2 or human RNA (for internal control) (A) Real-time analysis of CoV2-W3 primer set (targeting S gene) showed that LOD was 10 copies of RNA/assay. (B) End-point visualization of LAMP products with primer set CoV2-W3 using SafeBlue Illuminator/Electrophoresis System. (C) End-point visualization of LAMP products with primer set CoV2-W3 using a hand-held observation box with integrated blue LED and orange filter (Firebird Biomolecular Sciences LLC). (D) Real-time analysis of CoV2-v2-4 primer set (targeting N gene) showed an LOD of 25 copies of RNA/assay. (E) Real-time analysis of internal control RNaseP-2 primer set (targeting human RNase P gene) showed that LOD was 44 copies of human RNA/assay.

FIG. 6A. Sampling work-flow and results output. Dry nasal swabs are used as sampling method. Swabs are first eluted in a sample preparation buffer and aliquots from that are added into DP-RT-LAMP mixtures. End-point results are visualized by fluorescence of fluorescein excited by the emission of a blue LED and an orange filter. Saliva is mixed with a sample preparation buffer, and an aliquot is added into the DP-RT-LAMP mixture. End-point results are visualized using the same method for nasal swab sampling. Saliva is added to paper that has been covalently modified with quaternary ammonium salts (Q-paper). Q-paper carrying the saliva sample is directly introduced into DP-RT-LAMP mixture without further manipulation. End-point fluorescent signal is visualized using blue LED and orange filter. Note that the square of Q-paper is observable, but does not compromise the analysis.

FIG. 6B. In addition to end-point visualization, DP-RT-LAMP experiments were also run in real-time using Genie® II (Optigene, UK).

FIG. 7A-FIG. 7D. Optimization of sampling methods and fluorescence visualization with presently preferred methods. (A) Four different methods were evaluated for nasal swab sampling, including (i) TE elution, (ii) the method of Rabe-Cepko et al., (iii) a method combining Cepko with Chelex-100, and (iv) a process without a heating step. Heat-inactivated SARS-CoV-2 isolate was spiked into nasal swab elutions and each method's sensitivity was determined. Purified RNA control was included as a reference. (B) For saliva sampling, five methods were evaluated: (i) crude saliva without any treatment, (ii) the Cepko method, (iii) the Cepko method coupled with Chelex and a heat-step, (iv) the same without a heating step, and (v) deposition of saliva on Q-paper and its direct introduction into DP-RT-LAMP. A purified RNA control was included as a reference. (C) End-point visualization of finalized methods: Nasal swab and one of the saliva sampling methods uses buffer solution containing 1 mM Na citrate pH 6.5, 2.5 mM TCEP, 1 mM EDTA, 10 mM LiCl and 15% Chelex-100. LODs for both samplings were determined to be 100 copies/assay. (D) End-point visualization of saliva deposited on Q-paper and its direct use in DP-RT-LAMP reaction. The LOD was 100 copies/assay using Q-paper.

FIG. 8A-FIG. 8C. Further evaluation of presently preferred sampling methods and sensitivity analysis with contrived samples using heat-inactivated SARS-CoV-2 template from BEI. (A) Varying amounts of RNA was spiked into nasal swab samples. 500 copies of RNA were detected with consistency and RNase P gene was used as sampling control. (B) and (C) Varying amounts of RNA was spiked into saliva samples or saliva that was deposited onto Q-paper, respectively. 200 copies of RNA were detected with consistency and RNase P gene was detected successfully.

FIG. 9A-FIG. 9B. Analysis of inhibitory effects of saliva as a sample. DNA was used as the spike-in template and Tt values were determined in the absence (A) or presence (B) of saliva.

FIG. 10A-FIG. 10F. (A-C) Biplexed detection of SARS-COV-2 RNA and RNase P (internal control). Varying amounts (10⁵, 10⁴, 10³, 10², 10 and 5 copies) of heat-inactivated SARS-CoV-2 (BEI resources) spiked with 440 copies of purified human RNA. Fluorescence signals from three channels were recorded every 30 seconds using LightCycler® 480. Corresponding Tt values were shown on the table. Channel 483-533 (A and D) is specific for SARS-CoV-2 RNA, channel 523-568 (B and E) can detect signals from both targets (ladder formation manifests itself), and channel 558-610 (C and F) is specific for RNase P. (D-F) 10⁶ and 10³ copies of SARS-CoV-2 RNA was spiked into processed nasal swab samples and analyzed simultaneously using three fluorescent channels. Corresponding Tt values were shown on the table. The complexity of the curve when the competing viral amplification takes place involves a complex kinetic relationship as resources specific to the viral amplification (primer set) are consumed, allowing the positive control to again compete for common resources (e.g. dNTPs, enzymes).

FIG. 11A-FIG. 11C. (A) Workflow of lyophilization first involves the removal of glycerol from commercial enzymes. This is done by replacing enzyme storage buffer with its glycerol free version via ultrafiltration. The next step combines 10× primer mix and dNTPs with dialyzed enzymes. The mixture is then frozen (liquid N2) and lyophilized for 4-6 hours. (B) Lyophilized reagents were activated by supplementing lyophilized reagents with rehydration buffer, and templates containing SARS-CoV-2 RNA or contrived nasal/saliva samples were added to DP-RT-LAMP mixture was analyzed on Genie II and Tt values were determined. (C) End-point fluorescence was visualized using blue LED and orange filter.

FIG. 12. Artificially expanded genetic information system in a preferred embodiment.

FIG. 13. Self-avoiding molecular recognition system (SAMRS), in a preferred embodiment.

FIG. 14. A schematic showing, without limitation, the essential elements of a hand-held personal coronavirus detector. A cartridge (FIG. 15, for example) is inserted into the device. Pressing an “on button” starts a microprocessor to directs a heating element to maintain the liquid at 65±5° C. for a pre-selected period of time. During that time, an isothermal process amplifies an oligonucleotide, allowing the solution to allow the generation of unquenched fluorescence up excitation with a light. In this example, a blue 410 nm emission LED generates the fluorescence of fluorescein, whose fluorescence is observed through an orange filter. With a microprocessor of more sophistication, variable temperature and temperature cycling may optionally be performed.

FIG. 15. The cartridge used, without limitation, in the hand-held device in FIG. 14. The inventive steps include the use of Q-paper to receive saliva from the tongue of an individual who may be infected with the coronavirus; a sample of the saliva and the coronavirus that it may contain is thus delivered to the Q-paper. An inventive feature is to have optionally the Q-paper to carry a flavor, to encourage effective deposition of the saliva. A lid is then screwed on the tube, said lid carrying a protruding device that punctures a ball containing buffer required for the isothermal amplification processes. The bottom of the tube contains reagents that are required for the amplification, including without limitation, enzymes, oligonucleotides, and triphosphates. Also inventive is to incorporate into the device a thermosetting plastic that ensures that, after heating at 65±5° C. for a pre-selected period of time, the tube can no longer be opened.

FIG. 16. The processes of the instant invention may be executed in a kiosk that can deliver liquid. As an inventive step that simplifies workflow, the sample, including without limitation saliva, may be collected on a ball of size pre-selected to fit within a tube. In this implementation, the reagents are lyophilized in the tube, and liquid containing the other reagents is delivered by the kiosk. The kiosk has one or more slots that accept the tube after it receives a sample. The kiosk then delivers liquid to said tube, such that the combination of materials in the tube and in the kiosk-delivered liquid are sufficient to amplify the target DNA or RNA. The kiosk is further equipped with elements that warm a region of the tube to a temperature between 50 and 70° C., preferably 65° C. The amplification generates a fluorescent signal when viral RNA is present in the sample. A light in the kiosk illuminates the region of the tube that generates the fluorescent signal, and the kiosk contains an element that detects the fluorescent signal.

FIG. 17. The processes of the instant invention may optionally also be executed in a kiosk that can deliver liquid that is refrigerated so that the enzymes, triphosphates, and other unstable components required for the amplification are delivered by the kiosk. Other innovative features are described as elsewhere, including the collection of saliva on a ball of pre-selected size. That ball too may be optionally flavored, so as to encourage saliva collection. The ball may as well be semiporous, most preferably on its outer layers, to provide space into which saliva liquid carrying virus may enter. Also optionally, the ball may carry on or near its surface, material that selectively binds to the coronavirus, including without limitation an aptamer that binds the coronavirus, or an antibody that binds the coronavirus. In the latter case the liquid preferably washes the viral RNA from the surface of the tube at an elevated temperature.

### DESCRIPTION OF THE INVENTION

1. The Biochemistry that Generates Fluorescent Signals

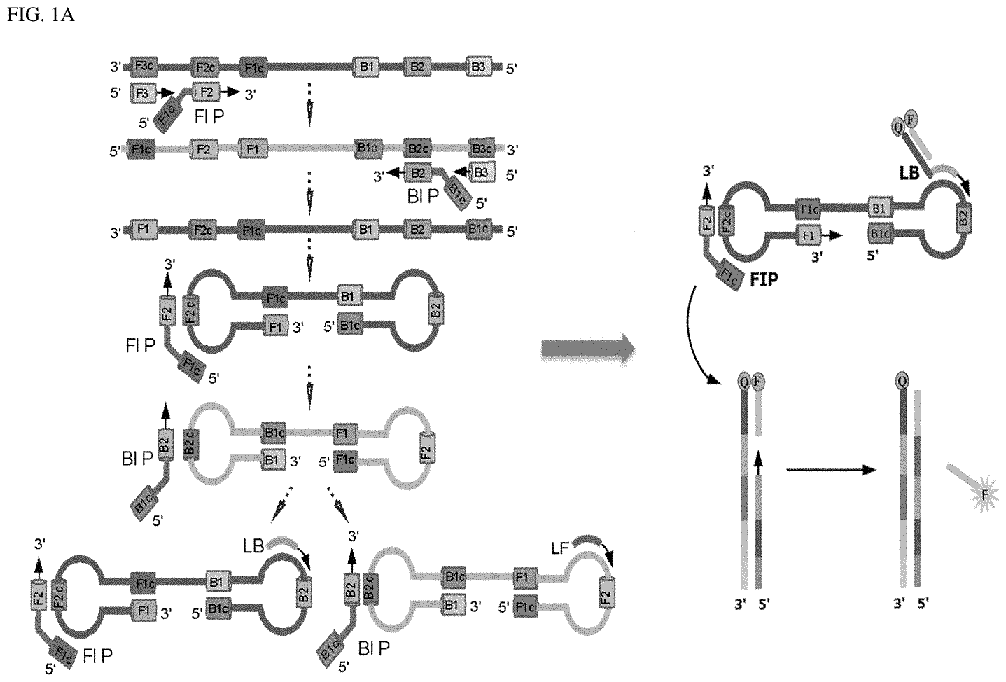

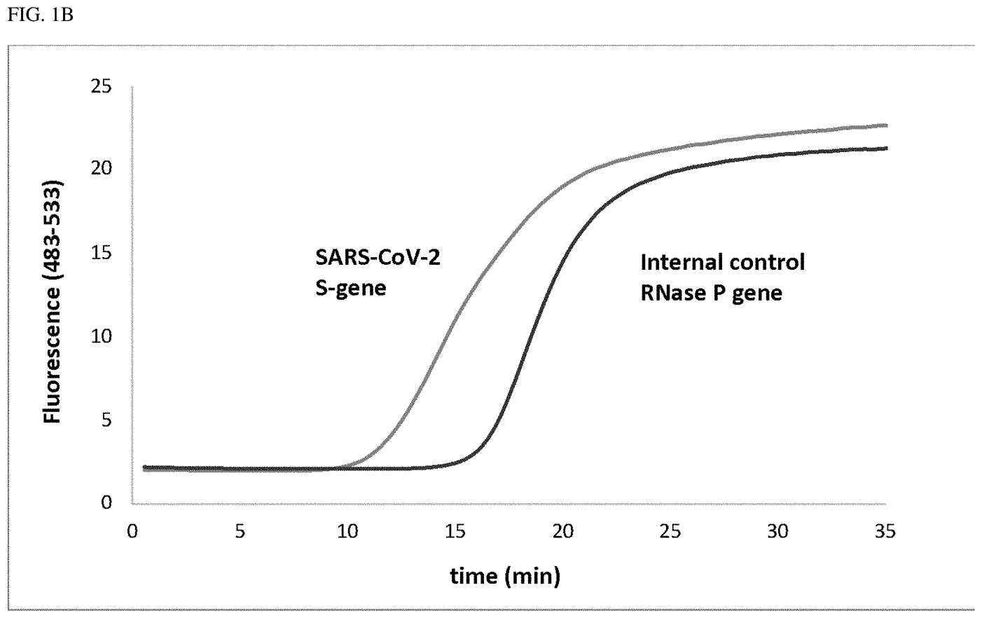

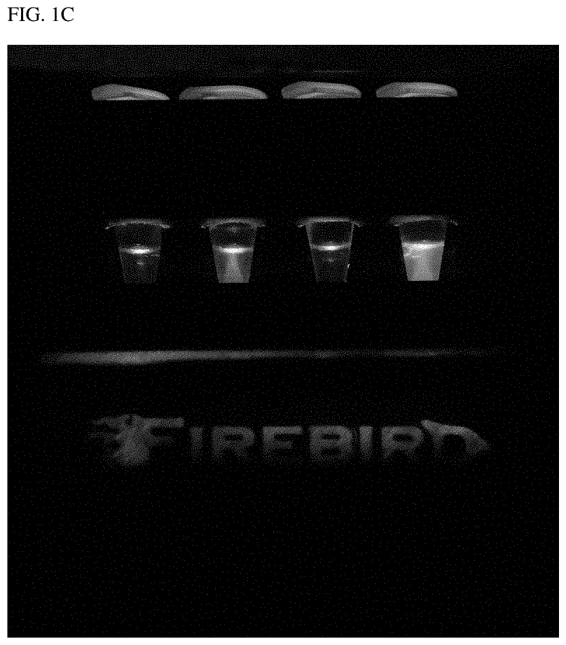

The displaceable probe LAMP (DP-LAMP), defined in U.S. patent application Ser. No. 15/826,126, is at the center of this invention. In its implementation to detect RNA viruses, it is initiated by a reverse transcriptase (RT), and is called here DR-RT-LAMP. In its classical form, DP-RT-LAMP uses six primers binding eight distinct regions within a target RNA (FIG. 1A). It runs at constant temperatures ranging from 62° C. to 72° C., a reverse transcriptase, and a DNA polymerase with strong strand displacing activity (most preferably, a Bst DNA polymerase). During the initial stages of DP-RT-LAMP, forward and backward internal primers (FIP and BIP), with outer forward and backward primers (F3 and B3), form a double loop structure. This becomes the seed structure for subsequent LAMP amplification. Amplification rate is further improved by the loop two primers (LF and LB), which are designed to bind the single stranded regions of the loops. These yield concatemers with multiple repeating loops [Nagamine K, Hase T, Notomi T. Accelerated reaction by loop-mediated isothermal amplification using loop primers. Mol Cell Probes. 2002; 16.].

In DP-RT-LAMP (as in DP-LAMP), signal is created by a displaceable probe, a short oligonucleotide carrying a 3′-fluorophore that is displaced from a complementary oligonucleotide as the desired amplification is completed. That complementary oligonucleotide has a 5′-quencher, and carries a tag that is a primer that binds to one of the loops in the initial LAMP double loop structure (FIG. 1A). Thus, each probe is delivered to the amplification mixture as a target-sequence-independent double-strand probe region and a single-stranded target-priming region.

In the displaceable probe architecture, in the absence of target, no fluorescence is observed due to quenching of fluorophore by a quencher in the undisplaced duplex. In the presence of target, the single-stranded portion of the quencher probe binds to the target and is extended. Further polymerase extension by reverse primers displaces the quencher strand from the fluorescently labeled strand, allowing the emission of fluorescence and its analysis in real-time. As a consequence of the displacing process, “S-shaped” curves appear in a plot of fluorescence versus time, similar to RT-PCR and similar Ct (or Tt-threshold time) analyses (FIG. 1B).

The displaceable probes can have sequences that have no substantial similarity to the sequence of any portion of the target analyte. This allows totally independent selection of the duplex sequence. This, in turn allows the fluorescently tagged displaced probe to be captured, either during or after the amplification. The fluor is preferably fluorescein (FAM), but any of a wide ranges of fluors known in the art may be used. Signal arising from the unquenched fluorescein in these particular displaced probes emerge in ca. 20 min and visible to human eye. Signals can be visualized in an observation box that uses a blue LED to excite the fluorescein, and an orange filter to allow the emission light to be observed without interferences with the excitation light (FIG. 1C). Fluorescence from other fluors, as known in the art, can be observed using excitation light and filters appropriate for other fluors.

Multiple fluors can be incorporated with multiple targets. In one implementation, FAM was used for SARS-CoV-2 detection and the internal control targeting the human RNase P RNA (λₑₓ-λₑₘ=495 nm-520 nm, color observed with excitation at 470 nm, green). JOE was used for RNase P gene for multiplexed LAMP experiments (λₑₓ-λₑₘ=529 nm-555 nm, excitation at 470 nm, yellow). Iowa Black FQ was used as a common quencher with absorption range of 420-620 nm. This quencher is often used with fluorophores that emit in the green to pink.

One process of the instant invention used first experiments sought to measure the sensitivity of a specific DP-RT-LAMP primer set (CoV2-W3) that had been selected from three trial sets that targeted the spike region of the virus genome. Here, RNA targets were prepared by transcription of a DNA template (˜230 nt). Varying concentrations of RNA was used to determine assay sensitivity [Glushakova, L. G., Barry W. Alto, B. W., Kim, M. S., Bradley, A., Benner, S. A. (2017) Detection of chikungunya viral RNA in mosquitoes on cationic (Q) paper based on innovations in synthetic biology. J. Virol. Methods 246, 104-111. PMC5967251] [Yaren, O., Bradley, K. M., Moussatche, P., Hoshika, S., Shu, Z., Karst, S. M., Benner, S. A. (2016) A norovirus detection architecture based on isothermal amplification and expanded genetic systems. J. Virol. Methods 237, 64-71. PMID: 27546345].

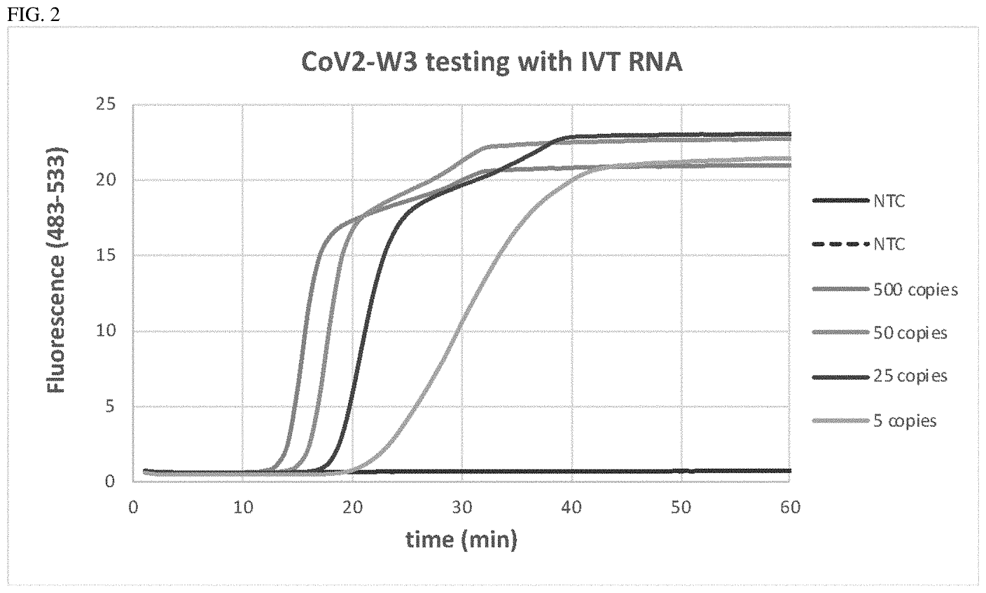

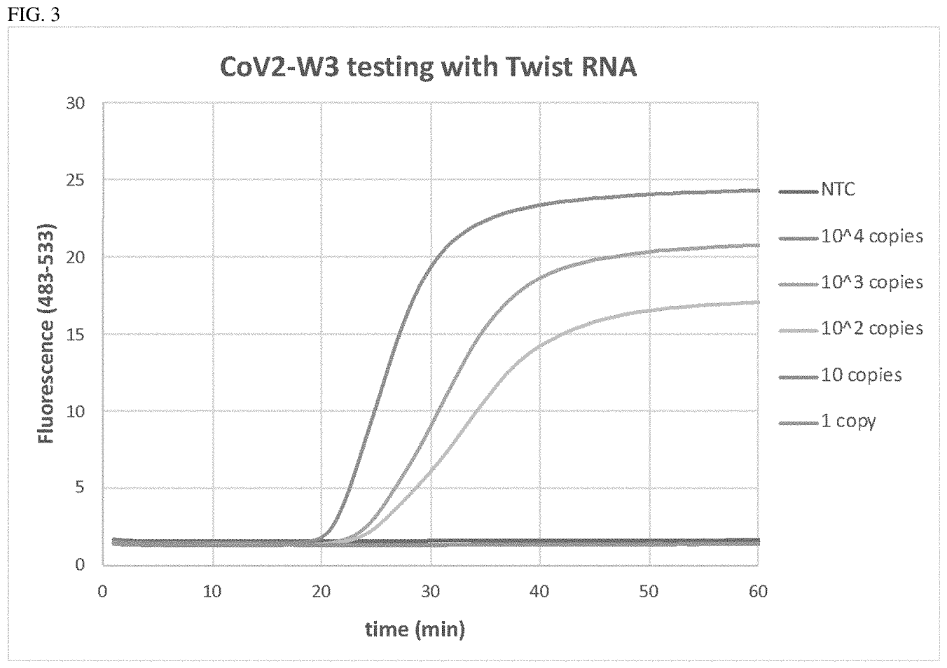

With this target, limits of detection (LODs) were 5 copies/assay, giving a threshold time (Tt, equivalent of Ct) of 22.5 min (FIG. 2, Table 1). When the 230mer RNA target was replaced by the complete RNA genome (Twist Biosciences, SARS-CoV-2 RNA), the sensitivity dropped to 100 copies/assay with Tt=25.3 min (FIG. 3, Table 1).

TABLE 1
Sensitivity measurements in initial conditions

| RNA target made by | | Time to | | | Time to |
|---|---|---|---|---|---|
| transcription of | | threshold, | RNA from | | threshold, |
| synthetic DNA | | Tt (min) | Twist | | Tt (min) |
| 500 | copies | 12.71 | 10000 | copies | 23.2 |
| 50 | copies | 14.87 | 1000 | copies | 24.3 |
| 25 | copies | 17.88 | 100 | copies | 25.3 |
| 5 | copies | 22.46 | 10 | copies | indefinite |

| | | | |
|---|---|---|---|
| NTC | indefinite | NTC | indefinite |

The sensitivity of the assay could be altered by changing conditions. These included:

(a) adding a second reverse transcriptase (SuperScript IV (SSIV) to the WarmStart reverse transcriptase (WS-RTx, NEB) already present.

(b) changing the reaction buffer,

(c) adding random hexamers (12 μM)

(d) adding excess reverse primer (B3 primer), and

(e) varying the incubation temperature (Table 2, Table 3)

TABLE 2
Improvement in the LOD using CoV2-W3 primer set and full-length
RNA template (Twist) by modifying DP-RT-LAMP conditions.

| Twist | | | | | | | | |
|---|---|---|---|---|---|---|---|---|
| RNA | | | | | | | | |
| copies | 10³ | 10² | 10 | NTC | 10³ | 10² | 10 | NTC |
| | 5x Buffer Excess B3 | | | | 10X Buffer Excess B3 only | | | |
| | SSIV-RT + WS-RTx | | | | WS-RTx 55° C. 10 min, | | | |
| | 55° C. 10 min, | | | | 65° C. 50 min | | | |
| | 65° C. 50 min | | | | | | | |
| Tt (min) | 16.6 | 19.3 | 37.1 | 40.6 | 17.3 | 19.9 | 18.8 | — |
| | 5x Buffer IDT RH | | | | 10X Buffer IDT RH only | | | |
| | SSIV-RT + WS-RTx | | | | WS-RTx 55° C. 10 min, | | | |
| | 55° C. 10 min, | | | | 65° C. 50 min | | | |
| | 65° C. 50 min | | | | | | | |
| Tt (min) | 19.3 | 21.2 | 24.3 | 46-48* | 20.1 | 27.2 | 29.3 | — |

*Sporadic NTC issue.

TABLE 3
Variety of DP-RT-LAMP components tested
with full length RNA target (Twist).

| | |
|---|---|
| W3_LAMP (S gene) | |
| Components | 1 Reaction 25 μL |

| | | | | |
|---|---|---|---|---|
| 5X Superscript IV Buffer | 5.0 | 5.0 | — | — |
| 100 μM B3 | 3.0 | — | 3.0 | — |
| 300 uM IDT RH (Random Hexamers) | — | 1.0 | — | 1.0 |
| 10X isothermal amplification buffer | — | — | 2.5 | 2.5 |
| 10 mM dNTPs with dUTP | 3.5 | 3.5 | 3.5 | 3.5 |
| 100 mM MgSO4 | 1.5 | 1.5 | 1.5 | 1.5 |
| 10X LAMP primers | 2.5 | 2.5 | 2.5 | 2.5 |
| Bst 2.0 warm-start NEB (8 U/μL) | 1 | 1 | 1 | 1 |
| WS-RTx NEB (15 U/μL) | 0.5 | 0.5 | 1 | 1 |
| RNase Inhibitor NEB (40 U/μL) | 0.5 | 0.5 | 0.5 | 0.5 |
| Superscript IV enzyme | 1.0 | 1.0 | — | — |
| Antarctic UDG NEB (1 U/μL) | 0.5 | 0.5 | 0.5 | 0.5 |
| 0.1M DTT | 1 | 1 | 1 | 1 |
| Twist RNA (1000, 100 and 10) or | 2 | 2 | 2 | 2 |
| background RNA for NTC | | | | |
| H₂O | 3.0 | 5.0 | 6.0 | 8.0 |

Samples were first incubated at 55° C. for 10 min, then at 65° C. for 50 min.

Each reaction mixture was pre-incubated at 55° C. for 10 min to ensure formation of sufficient cDNA by the warm start RT. This was then followed by incubation at 65° C.

These modifications improved sensitivity with the full-length RNA genome; LODs improved to 10 copies/assay. This compares favorably with SARS-CoV-2 colorimetric assay from New England Biolabs, which has a reported LOD of 500 copies/assay [Zhang Y, Odiwuor N, Xiong J, Sun L, Nyaruaba R O, Wei H, et al. Rapid Molecular Detection of SARS-CoV-2 (COVID-19) Virus RNA Using Colorimetric LAMP. medRxiv. 2020:2020.02.26.20028373. doi: 10.1101/2020.02.26.20028373]. However, use of 5× SSIV buffer gave fluorescent signal in the absence of target (no template controls, NTCs). This drove the choice of the presently preferred conditions that (i) use the original buffer, (ii) WS-RTx as the only reverse transcriptase, (iii) in the presence of random hexamers, and (iv) or excess B3. These conditions gave no “NTC problem” up to 60 minutes, with an LOD of 10 copies/assay. Refining these conditions further, better Tt values were observed with excess B3 than with random hexamers. Therefore, excess B3 primer was used in further DP-RT-LAMP experiments.

TABLE 4
Presently preferred conditions for the DP-RT-LAMP

| | |
|---|---|
| Condition 1 | NEB Buffer, excess B3, WS-RTx, 55° C. |
| | 10 min, 65° C. 50 min |
| Condition 2 | SSIV-RT Buffer, Random hexamer, |
| | SSIV-RT + WS-RTX, 55° C. |
| | 10 min, 65° C. 50 min |
| Condition 3 | NEB Buffer, WS-RTx, 65° C. 50 min |

Establishing DP-RT-LAMP Assay with High Sensitivity Using Heat-Inactivated Virus Isolate

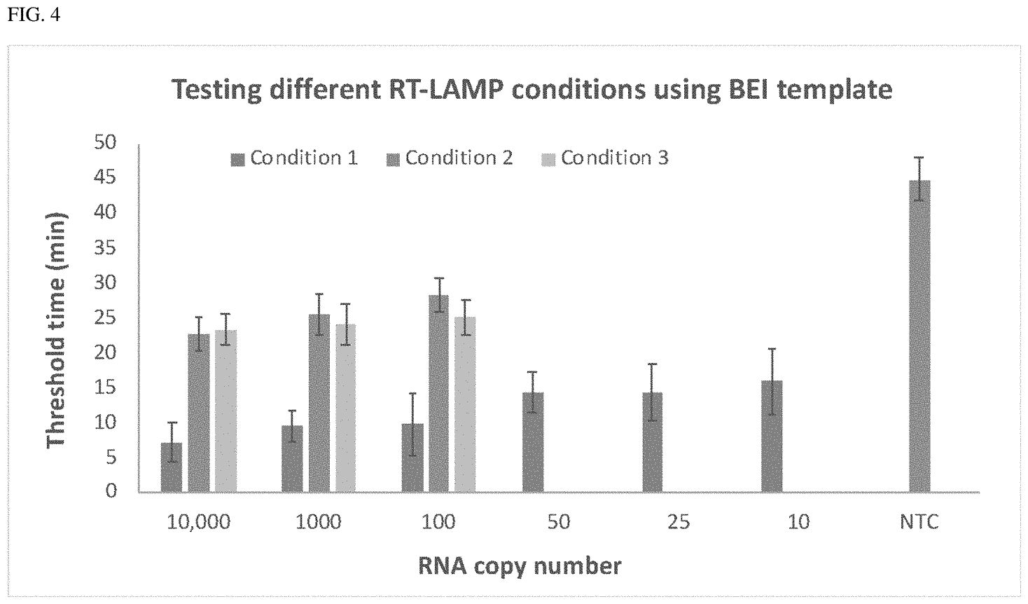

The LOD was assessed using authentic, non-synthetic virus that had been heat-inactivated (SARS-CoV-2 isolate, inactivated at 65° C. for 30 minutes, BEI Resources). This was also used to “spike” sample from nasal swabs and saliva samples. Three conditions previously tested for the synthetic RNA (Twist Bioscience) were again tested for the BEI target. The best sensitivity (10 copies/assay) was achieved with “Condition 1”, using the NEB isothermal amplification buffer, excess B3 primer, and WS-RTx with incubation at 55° C. for 10 min (initially), followed by further incubation at 65° C. 50 min (FIG. 4).

With the current modifications in DP-RT-LAMP protocol, another DP-RT-LAMP primer set was designed to target the N gene of SARS-CoV-2. We also designed a DP-RT-LAMP primer set to target the human RNase P gene. Detection of the amplicon from human RNAse P was intended to serve as an internal control to assess the adequacy of the sample collection.

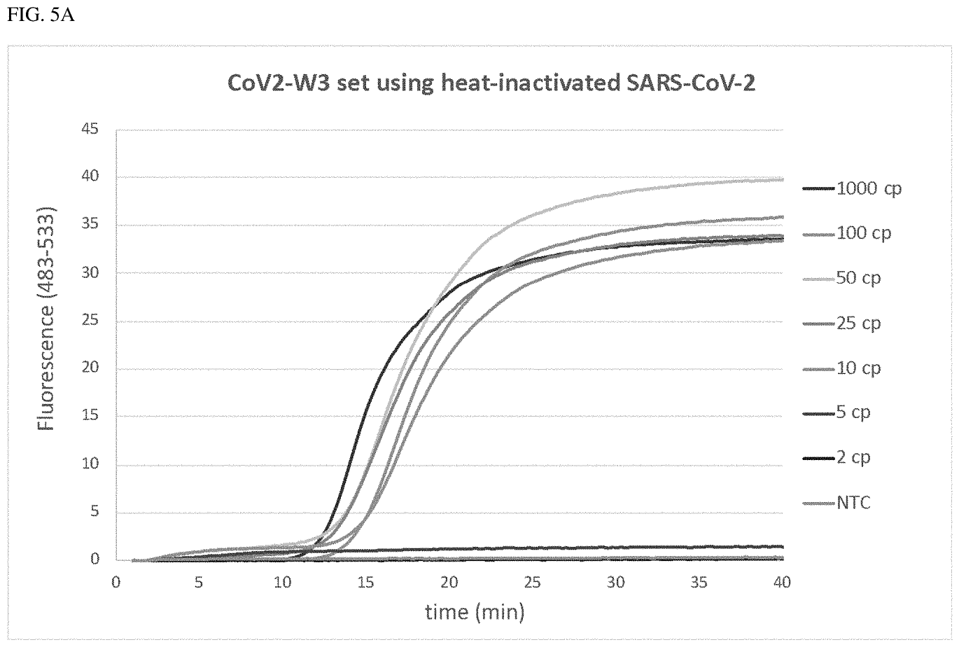

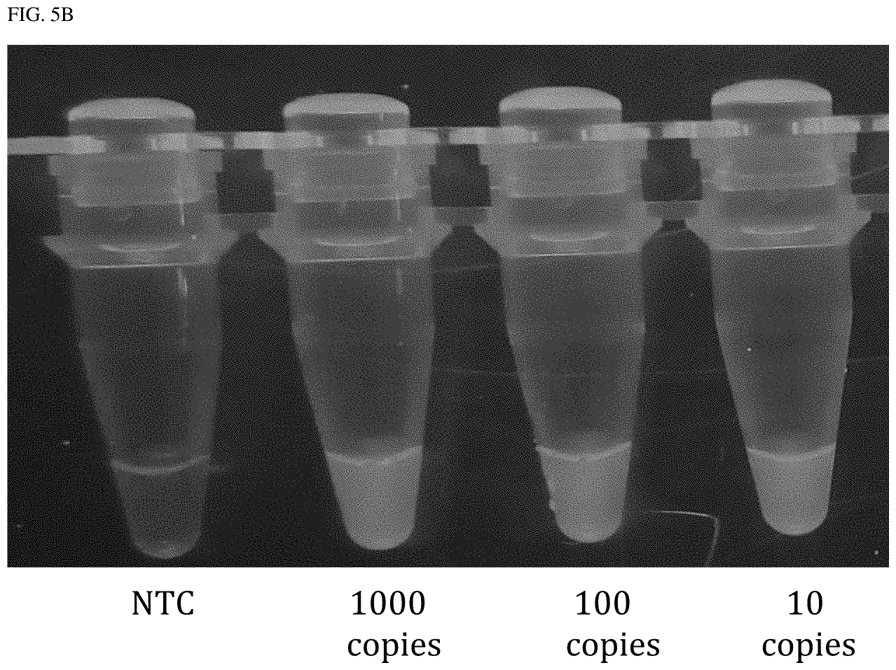

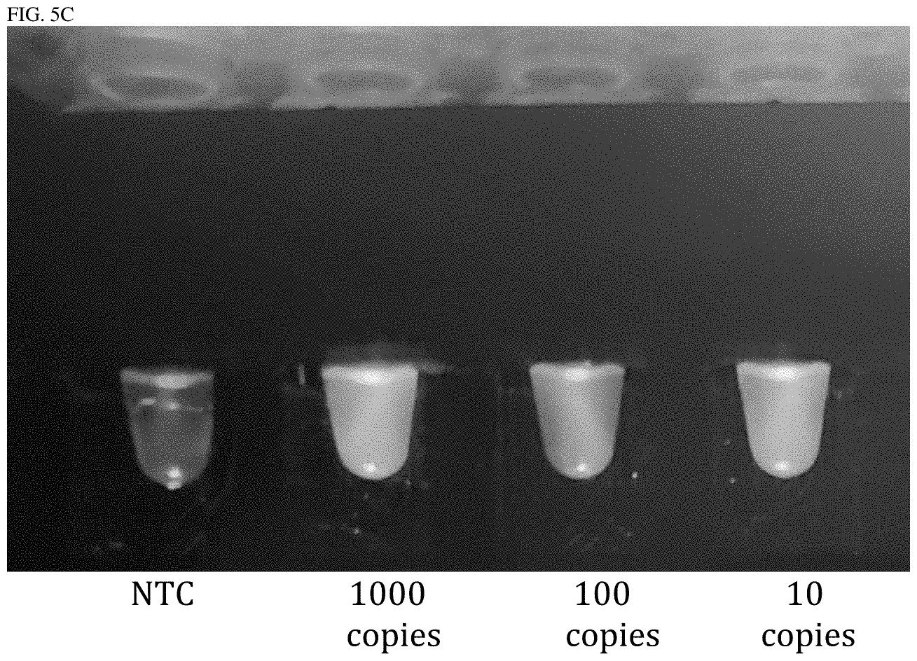

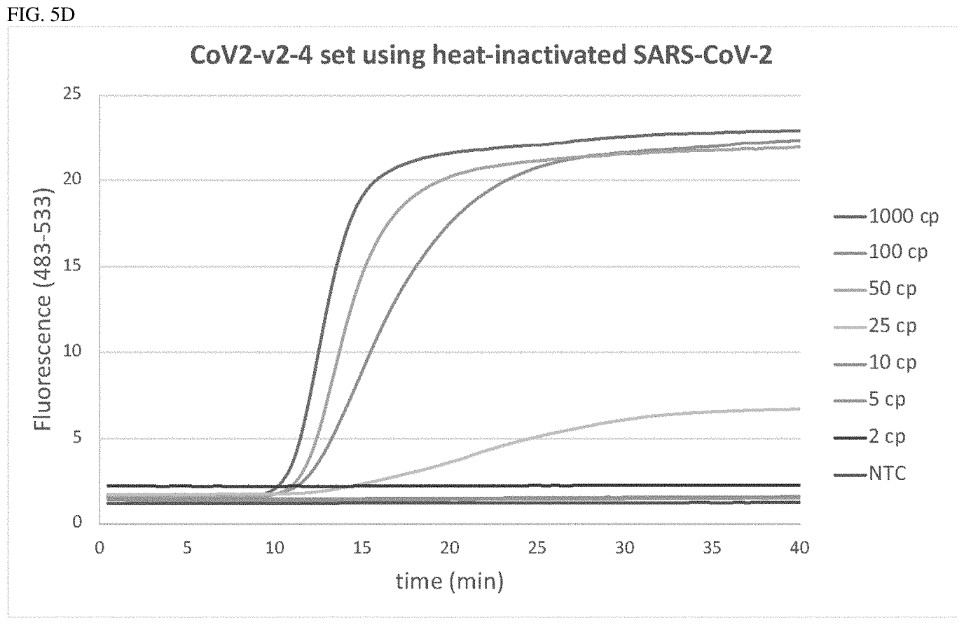

The primer set targeting S gene (CoV2-W3) gave an LOD of 10 copies/assay within 16 min; the fluorescence signal arising from fluorescence was excited at 470 nm (typically an LED) and visualized through an orange filter to block the excitation light (FIGS. 5A, 5B, and 5C). The primer set targeting the N gene (within the BEI sample) had an LOD of 25 copies/assay within a 12 min Tt. The system targeting the human RNase P gene had an LOD of 44 copies/assay, within a 16 min Tt (FIGS. 5D and 5E). Threshold times were compared to RT-qPCR where N gene and RNase P gene was detected in multiplex format. For this comparison, Ct values were converted to corresponding Tt values; DP-RT-LAMP was found to be very similar to multiplex RT-qPCR in terms of sensitivity, but was also faster than RT-qPCR. Comparative values are in Table 5 and

Table 6.

TABLE 5
Performance of DP-RT-LAMP for virus and human targets

| SARS-CoV-2 | | | | | | | | |
|---|---|---|---|---|---|---|---|---|
| RNA | | | | | | | | |
| (copy | Tt values (min) | | | | | | | |
| number) | 1000 | 100 | 50 | 25 | 10 | 5 | 2 | NTC |
| CoV2-W3 | 13.42 | 15.58 | 14.51 | 14.43 | 16.01 | No | No | No |
| | | | | | | signal | signal | signal |
| CoV2-v2-4 | 10.86 | 12.01 | 11.66 | 12.34 | No | No | No | No |
| | | | | | signal | signal | signal | signal |

Human RNA

| (copy | Tt values (min) | | | | | | | |
|---|---|---|---|---|---|---|---|---|
| number) | 4400 | 4400 | 440 | 440 | 44 | 44 | NTC | NTC |
| RNaseP-2 | 11.18 | 11.84 | 16.78 | 14.48 | 15.51 | 15.80 | No | No |
| (FAM) | | | | | | | signal | signal |

TABLE 6
Performance of standard RT-PCR for virus and human targets

| TaqPath ™ RT-qPCR | | | | | | |
|---|---|---|---|---|---|---|
| Mix | Ct values (corresponding Tt in min) | | | | | |
| RNA copies/reaction | 10,000 | 1000 | | 100 | 10 | NTC |
| N Gene | 26.7 (14.6) | 29.8 | (16.3) | 32.9 (18.1) | 36.9 (20.3) | No signal |
| RNase P Gene | 21.2 (11.6) | 24 | (13.2) | 28.1 (15.4) | 31.8 (17.5) | No signal |

### Simple Sample Preparation of Nasal Swabs and Saliva Samples

For a test to identify carriers who present a risk, sample preparation must be minimal, and instrumentation must be “field-deployable”. Several have sought low sample preparation workflows, as RNA purification from biological samples is time consuming and timely delivery of test results can be impaired due to limited supplies of sample purification kits [Rabe B A, Cepko C. SARS-CoV-2 Detection Using an Isothermal Amplification Reaction and a Rapid, Inexpensive Protocol for Sample Inactivation and Purification. medRxiv. 2020:2020. 04.23.20076877] [Bhadra S, Riedel T E, Lakhotia S, Tran N D, Ellington A D. High-surety isothermal amplification and detection of SARS-CoV-2, including with crude enzymes. bioRxiv. 2020:2020.04.13.039941][Joung J, Ladha A, Saito M, Segel M, Bruneau R, Huang M-1W, et al. Point-of-care testing for COVID-19 using SHERLOCK diagnostics. medRxiv. 2020:2020.05.04.20091231.][Kubota K, Jenkins D M, Alvarez A M, Su W W. Fret-based assimilating probe for sequence-specific real-time monitoring of loop-mediated isothermal amplification (LAMP). Biol Eng Trans 2011; 4:81-100][Anahtar M N, McGrath G E G, Rabe B A, Tanner N A, White B A, Lennerz J K M, et al. Clinical assessment and validation of a rapid and sensitive SARS-CoV-2 test using reverse-transcription loop-mediated isothermal amplification. medRxiv. 2020:2020.05.12.20095638][Dao Thi V L, Herbst K, Boerner K, Meurer M, Kremer L P M, Kirrmaier D, et al. Screening for SARS-CoV-2 infections with colorimetric RT-LAMP and LAMP sequencing. medRxiv. 2020:2020.05.05.20092288].

To meet these specs, we generated three protocols for SARS-CoV-2 testing. The behavior of the virus itself defines the sampling procedure. False negatives arising from defective sampling are often as problematic as (or more problematic than) false negatives arising from failure of the assay. Fortunately, the life cycle of SARS-CoV-2 appears to allow simple sampling, with even mid-turbinate sampling being adequate, as well as saliva sampling [Broughton J P, Deng X, Yu G, Fasching C L, Servellita V, Singh J, et al. CRISPR—Cas12-based detection of SARS-CoV-2. Nature Biotechnology. 2020; 38(7):870-4.] [Srivatsan S, Han P D, van Raay K, Wolf C R, McCulloch D J, Kim A E, et al. Preliminary support for a “dry swab, extraction free” protocol for SARS-CoV-2 testing via RT-qPCR. bioRxiv. 2020:2020.04.22.056283].

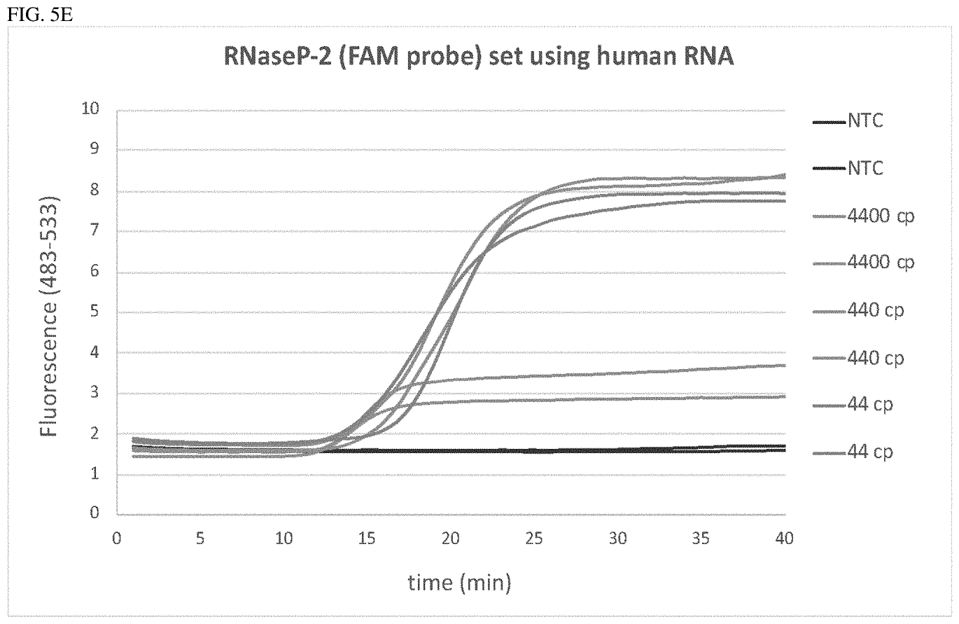

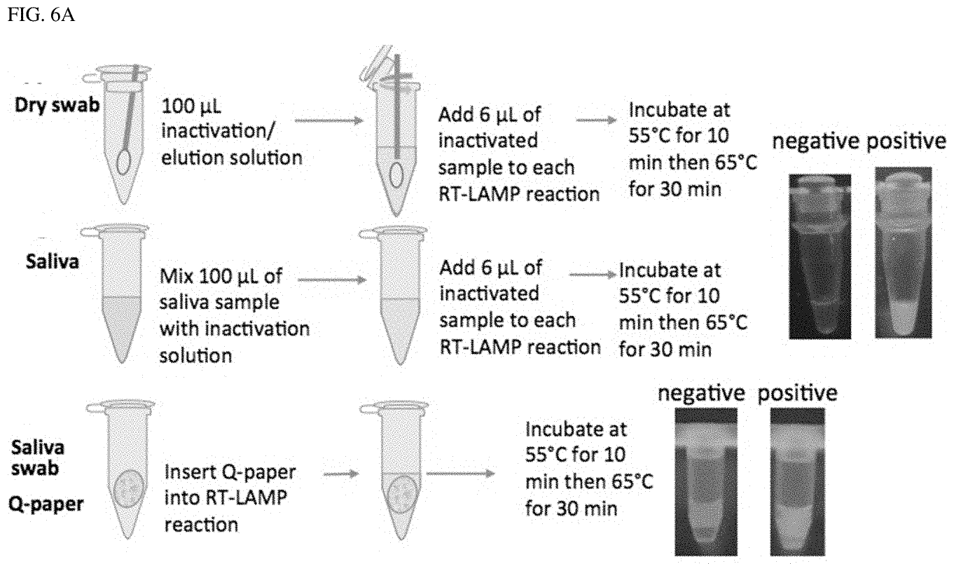

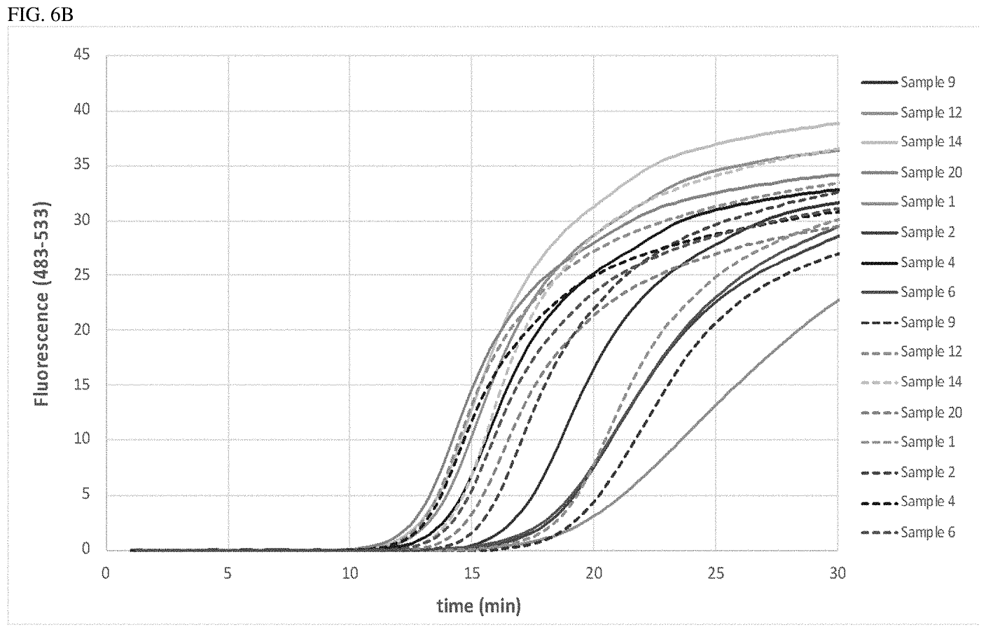

Therefore, a preferred protocol uses dry mid-turbinate or anterior nasal swabbing as a collection method, and relies on the positive control targeting human RNase P to ensure that the collection was adequately aggressive. Post sampling, swabs were eluted in various elution/inactivation buffers. An aliquot from the elution solution was added directly to the DP-RT-LAMP mixture, and analyzed in real-time and by visualization of end-point fluorescence (FIG. 6A). Spiked saliva (with saliva alone as the negative control) diluted with concentrated inactivation buffer (1:100 ratio of buffer to saliva) was also used. An aliquot of the resulting mixture was added to the DP-RT-LAMP mixture and analyzed similarly (FIG. 6A).

Alternatively, saliva can be placed on “Q-paper”, a cellulose filter paper that carries quaternary ammonium groups. Q-paper has been previously used to capture arboviral RNA from single mosquitoes after a drop of ammonia is added to the carcasses [Yaren O, Alto B W, Gangodkar P V, Ranade S R, Patil K N, Bradley K M, et al. Point of sampling detection of Zika virus within a multiplexed kit capable of detecting dengue and chikungunya. BMC Infectious Diseases. 2017; 17(1):293. doi: 10.1186/s12879-017-2382-0]. In this work, the Q-paper holding the viral RNA could be added directly to the DP-RT-LAMP mixture without any sample preparation. The fluorescence can be analyzed in real-time or by end-point visualization, again using blue LED excitation with fluorescence observed through an orange filter (FIG. 6A). The fluorescence can also be seen in a hand-held observation box.

Real-time analyses of all methods tested were performed on a (Roche LightCycler® 480). However, performance was equally satisfactory when light readout was done on a portable Genie® II instrument, available from Optigene. Genie® II processes 16 samples simultaneously using the FAM-channel (483-533 nm). The data outputs are similar to those obtained with the more expensive real-time PCR instrument. Genie® II offers positive/negative results with Tt values as good as obtained with the PCR instrument, but at a fraction of the cost and useable in the lobby of a workplace, a courtroom, or a school (FIG. 6B).

Presently preferred workflows for 2019-nCoV are, with presently preferred volumes:
(a) Dry nasal swabs are the sample method, obtained by mid-turbinate swabbing.
(b) Swabs are eluted in a sample preparation buffer, typically 50-1000 μL.
(c) An aliquot (typically 4 to 10 μL, maximum 25 μL) from the sample preparation buffer holding the eluate is added to a DP-RT-LAMP mixture.
(d) The mixture is heated at 65° C. for 15-30 minutes.
(e) End-point results are visualized using blue LED and orange filter.

As an alternative presently preferred workflow, saliva (typically 100-1000 μL) is spit into a tube, and a sample (typically 5-10 μL) is added directly to a portion of sample preparation buffer briefly. Here, aliquot of that mixture is added to the DP-RT-LAMP mixture (typically 25-100 μL). End-point results are visualized as with the nasal swab samples.

As an alternative presently preferred workflow, saliva (typically 10-50 μL) is placed onto a small square (typically 3-5 mm) of quaternary ammonium modified paper (Q-paper). The Q-paper coated with saliva is directly introduced into DP-RT-LAMP mixture (typically 50-200 μL) without further manipulation. End-point fluorescent signal is visualized using blue LED and orange filter, as before. The Q-paper square is observable, but does not hinder the analysis.

As an alternative presently preferred workflow, to replace end-point visualization, DP-RT-LAMP experiments are also run in real-time using a Genie® II (Optigene, UK) instrument. This allows the appearance of fluorescence arising from the displaced probes to be visualized as the amplification proceeds. Representative curves are shown in various drawings.

Validation of DP-RT-LAMP Assay with Contrived Nasal Swabs

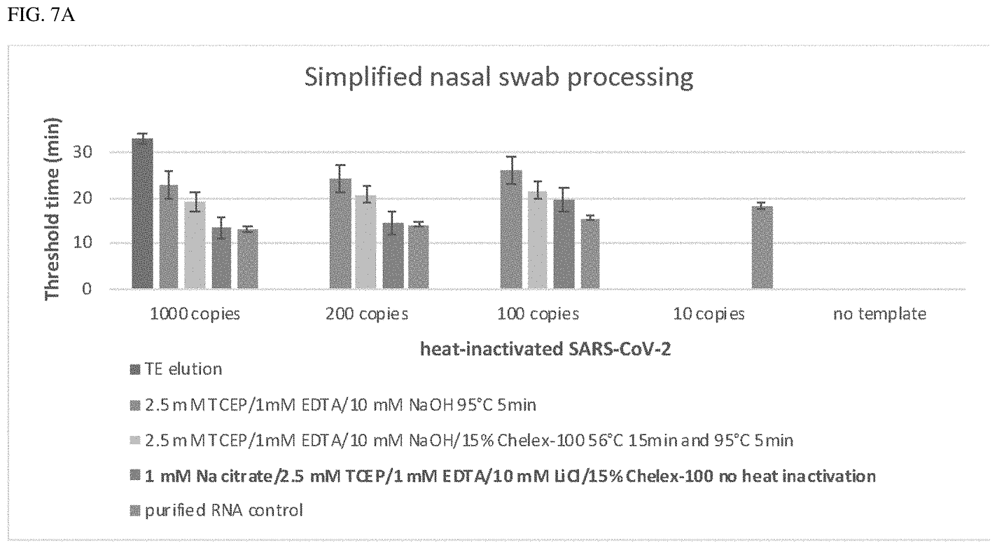

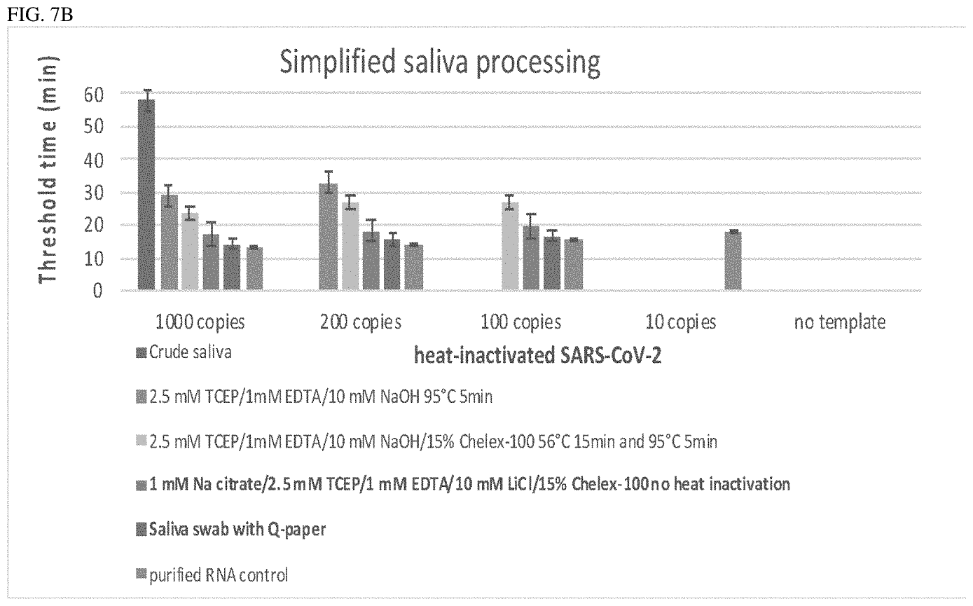

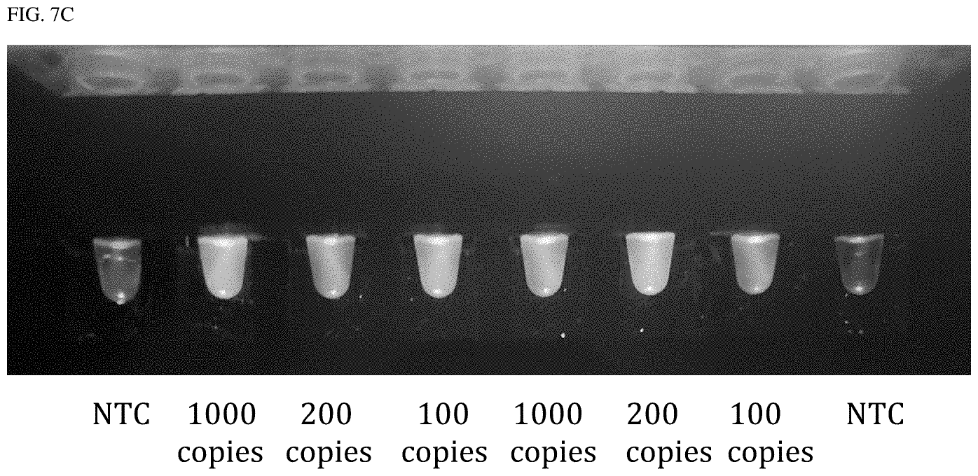

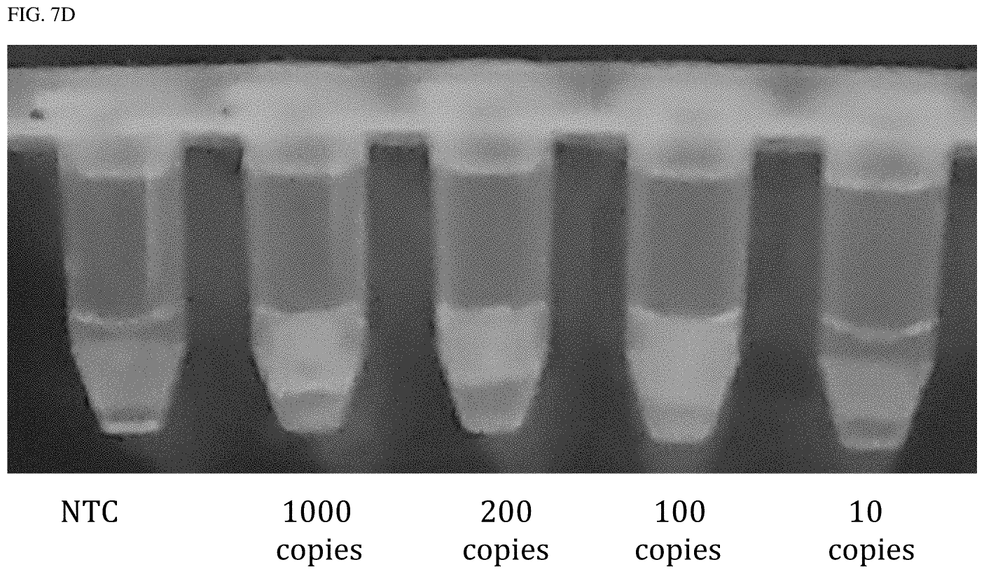

Having established work-flow parameters, we tested various elution/inactivation buffers with or without a heat step to design the presently preferred protocol. FIG. 7A summarizes the methods used to process mid-turbinate or nasal anterior swabs. TE (Tris-HCl pH 7.0 and 1 mM EDTA) as an elution buffer gave LODs≈1000 copies/assay, with Tt values of ≈30 min. The procedure of Rabe and Cepko [op. cit.] was used, with swabs eluted in buffer containing NaOH, TCEP and EDTA and incubated at 95° C. for 5 min, and then spiked with known concentrations of BEI template. These gave LODs as low as 100 copies/assay, with a Tt of 23 min.

Despite its promise, this approach did not give reproducible results when nasal swabs were spiked with inactivated virus prior to the 95° C. heating step. A similar problem was observed when same buffer was combined with Chelex-100, in a workflow that incorporated two heating steps, one at 56° C. for 15 min and a second at 95° C. for 5 min. Dao Thi et al. [op. cit.] also report similar results when nasal swab elution mixtures were spiked with RNA, and then heated (95° C., 5 min). However, treatment of clinical samples using the method developed by Rabe and Cepko [op. cit.] with heating at 95° C. for 5 min did not cause a decrease in assay sensitivity.

To further simplify sampling work-flow, NaOH was replaced with sodium citrate (in a pH range of 5 to 7, preferably pH 6.5) and added TCEP, EDTA, LiCl and Chelex-100. Swabs were eluted at room temperature without any additional heating step. Here, 100 copies of viral RNA were detectable per assay within 16-18 min. In addition to real-time analysis, end-point fluorescent images were also visible to human eye at 100 copies/assay (FIG. 7C).

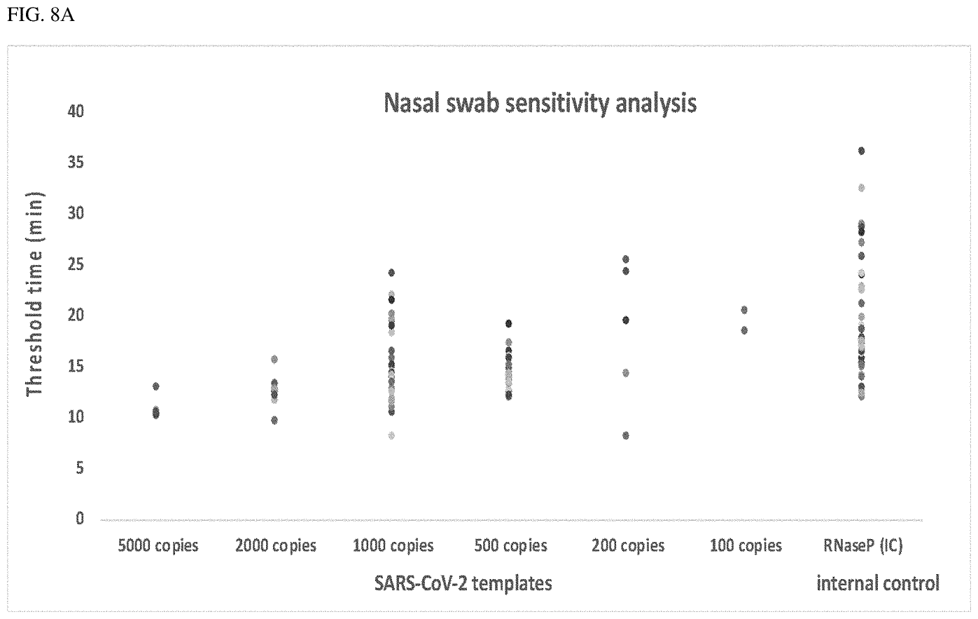

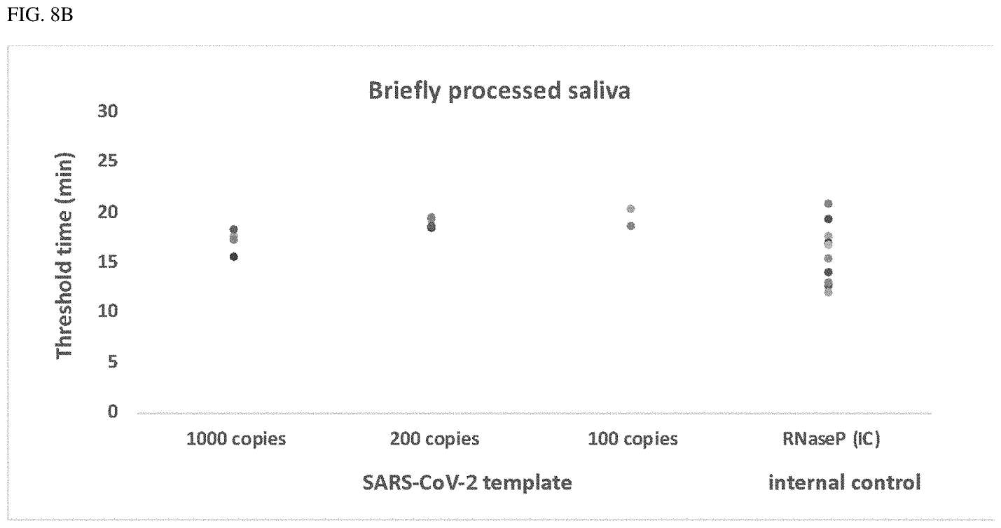

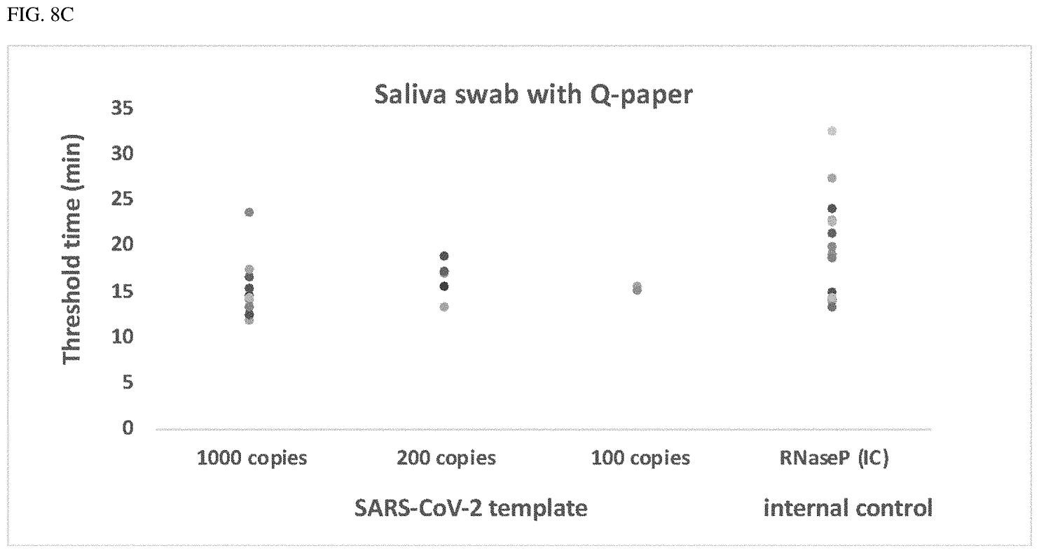

The sensitivity with nasal swab samples was analyzed using contrived nasal swabs. Here, 500 RNA copies/assay were detected consistently at 100%. Ca. 200 copies/assay were detected with 50% efficiency, and 100 copies of RNA/assay were detected at 20% efficiency. The internal control that targets the RNase P gene was detected at 100%, indicating that the sample collection was sufficiently aggressive (FIGS. 8A and 8D).

Validation of DP-RT-LAMP Assay with Contrived Saliva Samples

Crude saliva was first added to DP-RT-LAMP without any treatment, with a saliva:LAMP reaction mixture ratio of 1:5. As shown in FIG. 7B, 1000 copies of RNA were detectable only after 50 min. This suggested that crude saliva was not useable as a sample on its own.

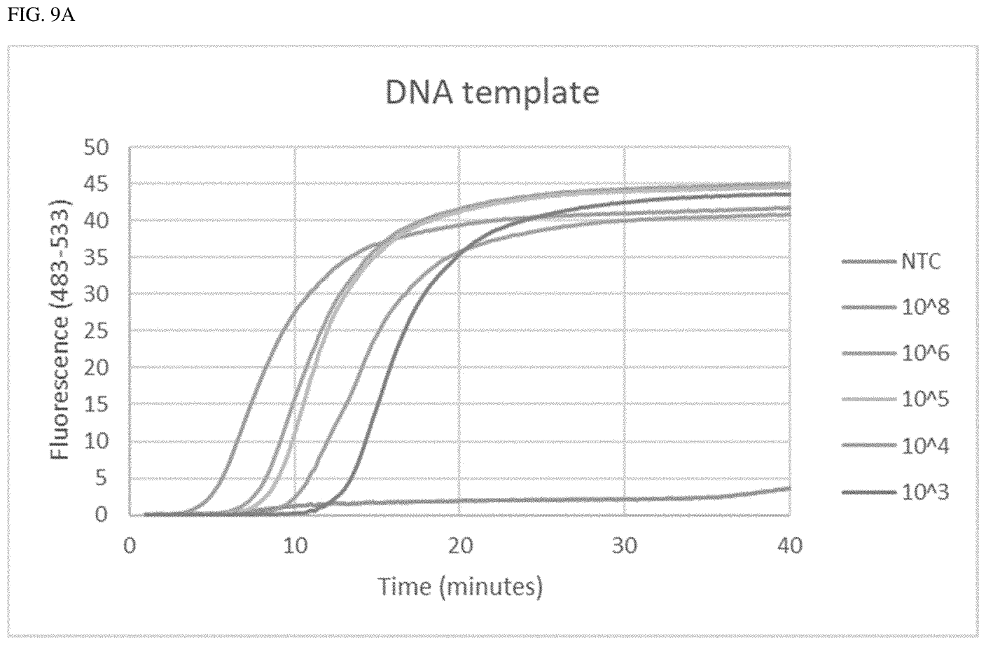

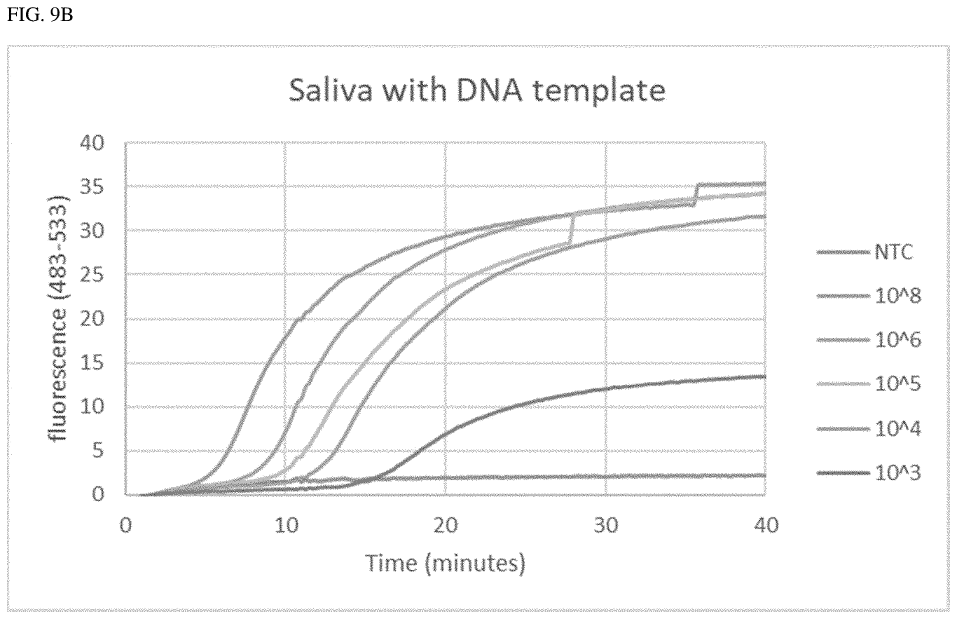

Suspecting that RNA might be rapidly degraded in saliva, saliva samples spiked with DNA were tested (FIG. 9). Here again, the emergence of the signal was substantially delayed, even though the delay was not as large as with the analogous RNA. We then tried nasal elution buffers in more concentrated form. Here, saliva (100 μL) was treated with 100× buffer (0.25 M TCEP, 0.1 M EDTA, 0.1M NaOH or Na citrate, 1 μL) with or without 15% Chelex-100, and with or without a heating step. Multiple runs showed the most reliable results with inactivation buffer containing TCEP, EDTA, sodium citrate, LiCl and Chelex-100; the LOD was ˜100 copies/assay. Fluorescent signals obtained from positive samples were clearly differentiable from those arising from samples lacking target (FIG. 7C).

As an alternative to this saliva sampling method, saliva was absorbed on to Q-paper, which was placed after a brief time (5 min) at room temperature (to simulate how processing might occur in a workplace lobby) directly into the DP-RT-LAMP mixture. Analogous to what is seen with mosquito carcasses [Yaren et al. 2017, op. cit.], 100 copies of viral RNA were detected by spotting SARS-CoV-2 RNA onto saliva coated Q-paper and directly amplified by DP-RT-LAMP. Visualization of positive signals was done in the 3-D printed box (FIG. 7D).

The sensitivities of the assay with two saliva sampling methods were further analyzed using contriving saliva samples. Here, 200 copies of RNA/assay could be detected by both methods with 100% detection rate with a small sample size (5 cases); 100 copies of RNA were detected with 50% efficiency using 100× inactivation solution, and with 40% efficiency using Q-paper for sampling. Additionally, the internal control targeting RNase P gene was detected at 100% in both methods (FIG. 8A-C).

TABLE 7
Performance of assay with whole coronavirus (BEI Resources)

| SARS-CoV-2 | | | | | | | |
|---|---|---|---|---|---|---|---|
| RNA copies/assay | 5000 | 2000 | 1000 | 500 | 200 | 100 | RNase P (IC) |

Nasal swabs

| | | | | | | | |
|---|---|---|---|---|---|---|---|
| mean Tt (min) | 11.55 | 13.2 | 16.01 | 14.4 | 18.2 | 19.4 | 19.4 |
| (Positive/Total) | (5/5) | (10/10) | (32/32) | (23/23) | (5/10) | (2/10) | (44/44) |

| | | | | |
|---|---|---|---|---|
| SARS-CoV-2 RNA | | | | |
| copies/assay | 1000 | 200 | 100 | RNase P (IC) |

Briefly processed saliva

| | | | | | |
|---|---|---|---|---|---|
| mean Tt (min) | 17.03 | (5/5) | 18.9 (5/5) | 19.3 (2/4) | 18.9 (10/10) |
| (Positive/Total) | | | | | |

Saliva swabs using Q-paper

| | | | | | |
|---|---|---|---|---|---|
| mean Tt (min) | 15.5 | (5/5) | 15.8 (5/5) | 15.3 (2/5) | 20.3 (15/15) |
| (Positive/Total) | | | | | |

TABLE 8
Variety of DP-RT-LAMP components tested with full length RNA target (Twist).

| | Threshold time (min) | | | | | | |
|---|---|---|---|---|---|---|---|
| CoV2-W3 | 10⁸ | 10⁶ | 10⁵ | 10⁴ | 10³ | 10² | No |
| primer set | copies | copies | copies | copies | copies | copies | template |
| DNA only | 6.13 | 8.57 | 9.56 | 11.15 | 14.14 | Not run | No signal |
| Saliva spiked | 7.14 | 10.24 | 12.05 | 14.08 | 17.15 | Not run | No signal |
| with DNA | | | | | | | |

### Multiplex Detection of SARS-CoV-2 and RNase P

An assay robust for workplace entrance use must incorporate a signal to indicate that sampling is sufficiently aggressive. The DP architecture allows simultaneous detection of viral RNA and human RNase P gene in single tube. To show this, we spiked varying amounts of viral RNA into 440 copies of purified human RNA. Using the LightCycler® 480 and three fluorescent channels, 10 copies of SARS-CoV-2 RNA could be detected in the presence human RNA in two-plex format when equal amount of the two (virus and human) 10× LAMP primer sets were present.

When viral RNA was present in higher amounts, the signal for the positive control was delayed to 32.5 minutes, instead of appearing 21-23 min. This is presumed to reflect the two amplification processes competing for some of the same LAMP amplification resources.

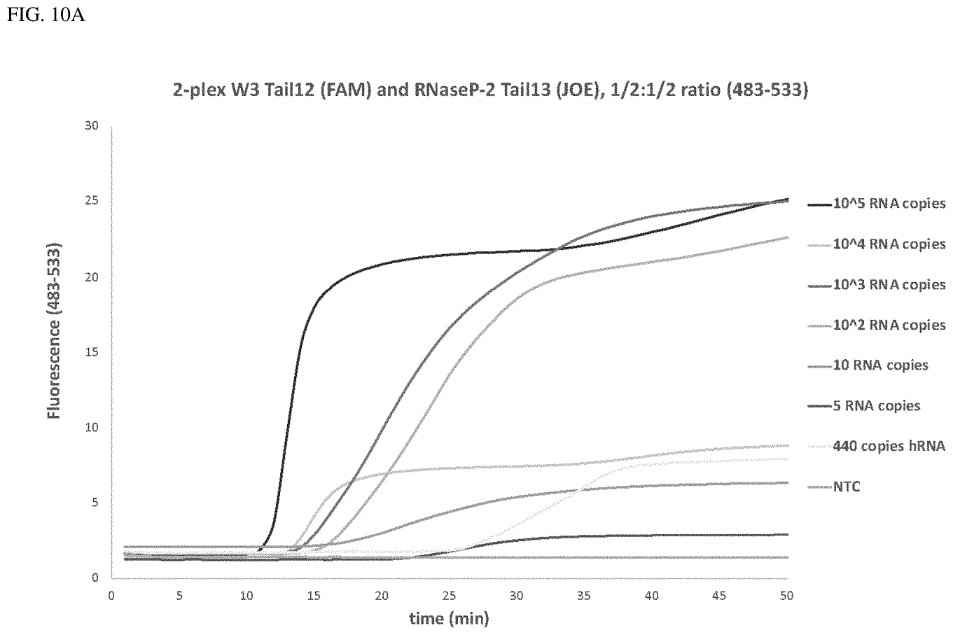

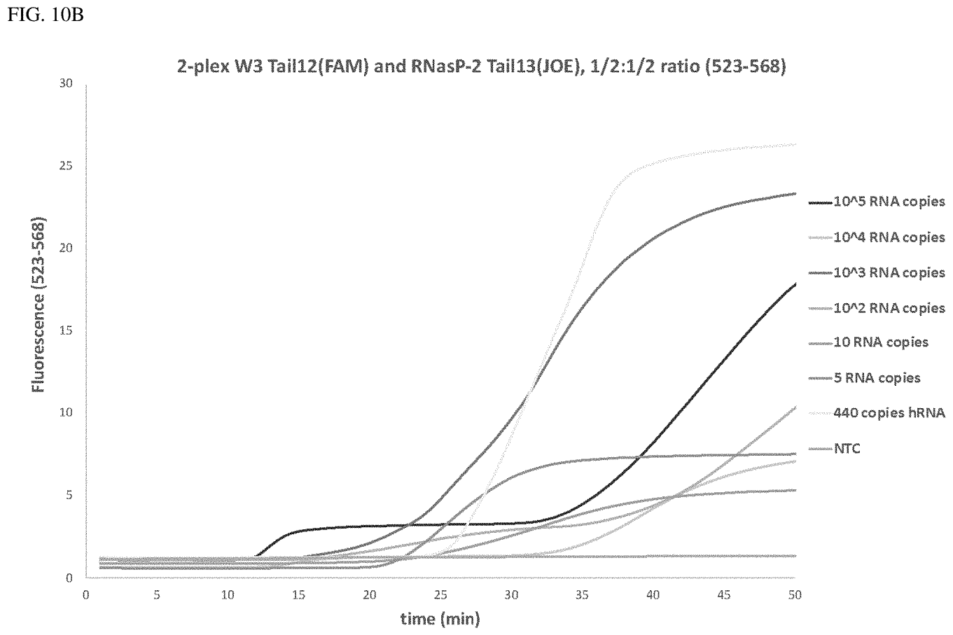

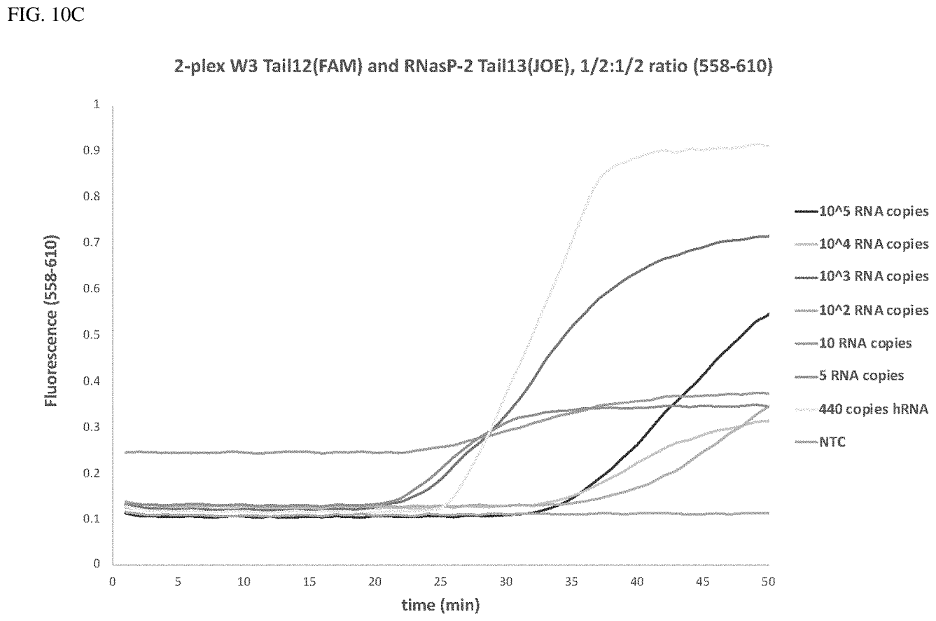

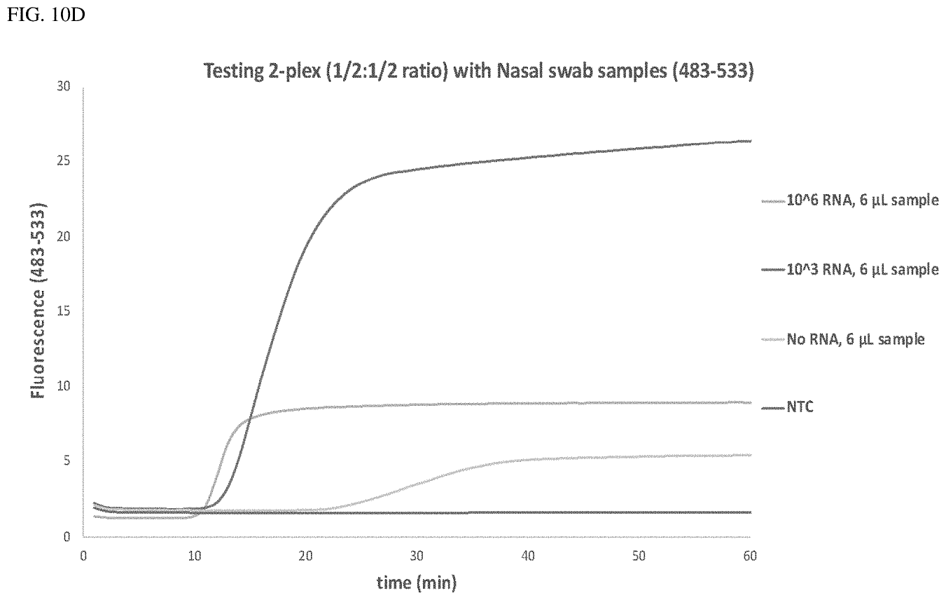

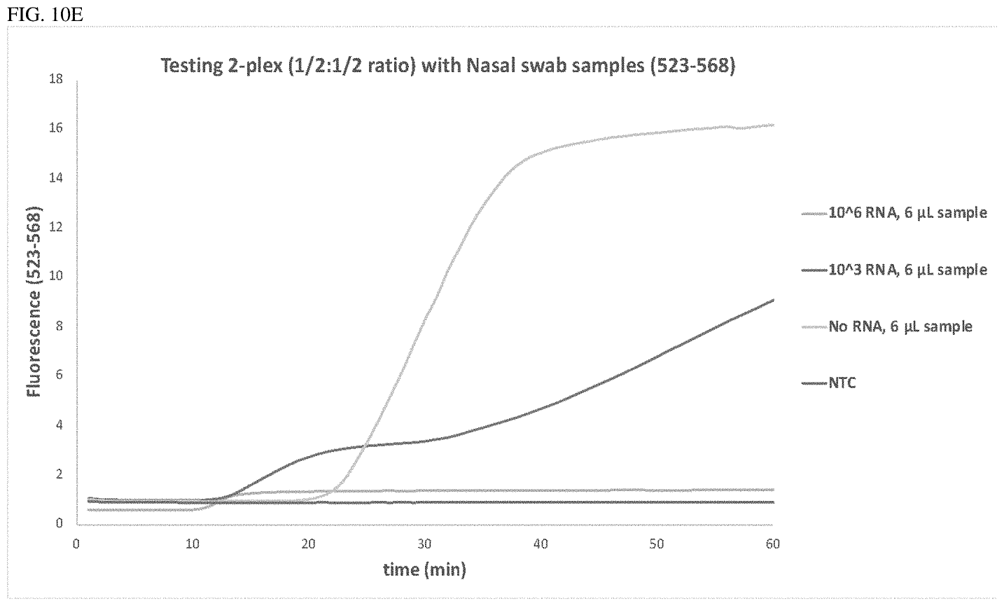

A similar degree of sensitivity was achieved when viral RNA was ˜1000 copies/assay (FIG. 10A-C). We spiked 10⁶ and 10³ copies of viral RNA into processed nasal swab samples and ran multiplexed assays. When 10⁶ copies of RNA were present, the RNase P gene was not detected, since the viral amplifications consumed LAMP resources (e.g. dNTPs). Thus, the presence of the virus signal itself proves that the LAMP amplification is working; a separate positive control is not needed. In the presence of 1000 copies of RNA, both targets were detected efficiently with Tt value of RNase P being very similar to its value without viral RNA (FIG. 10E-F).

### Lyophilization of DP-RT-LAMP Reagents

For workplace entry use, the reagents used must robustly survive transport and storage in amateur hands. Accordingly, lyophilized reagent mixtures were prepared and their performance tested.

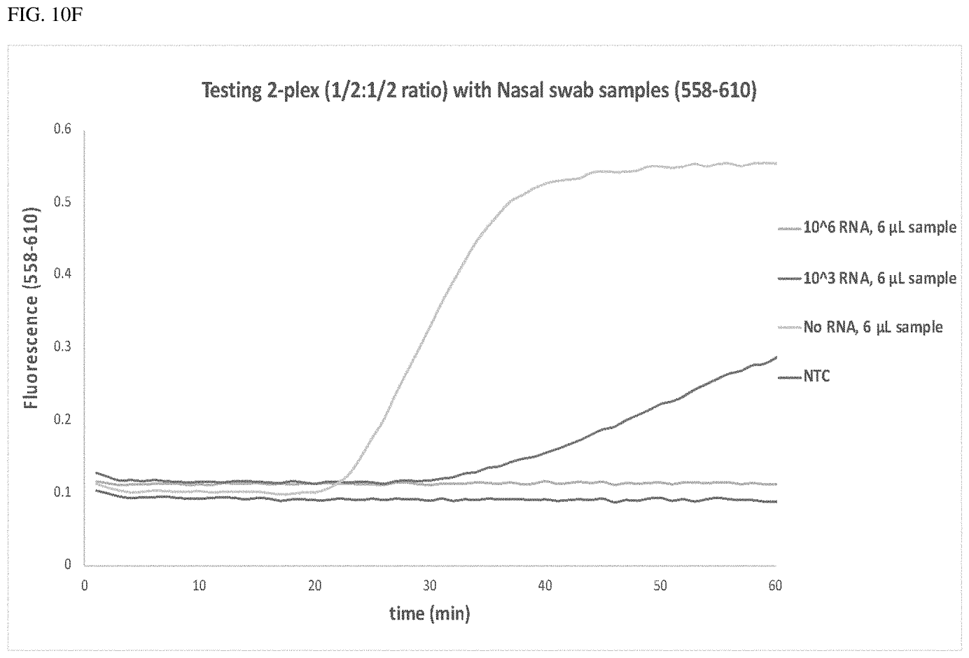

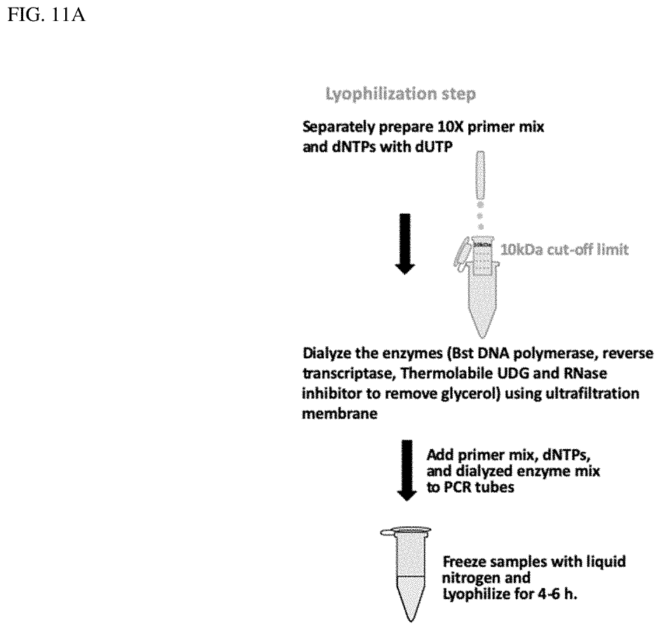

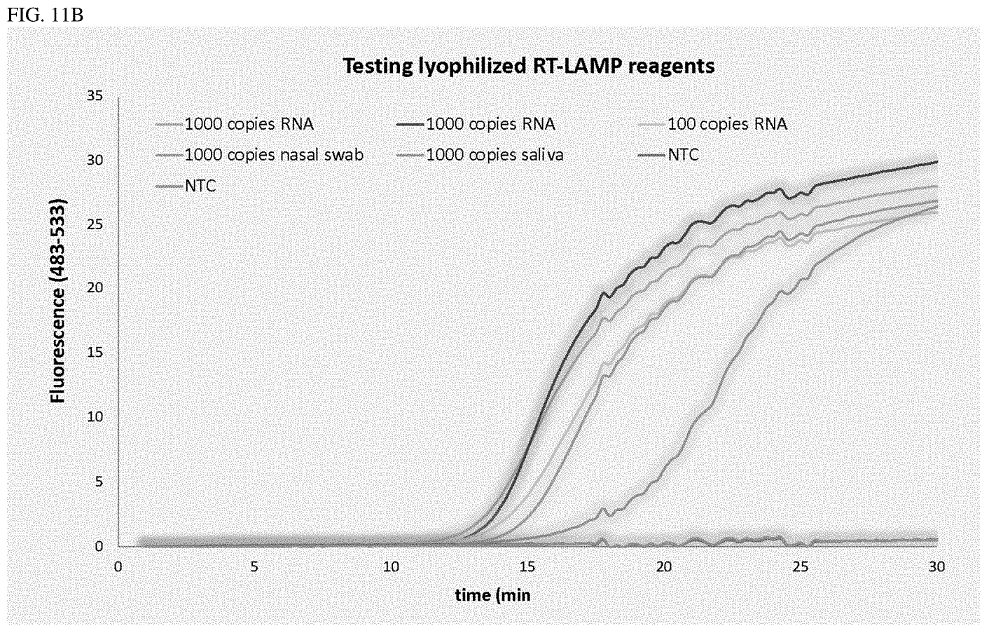

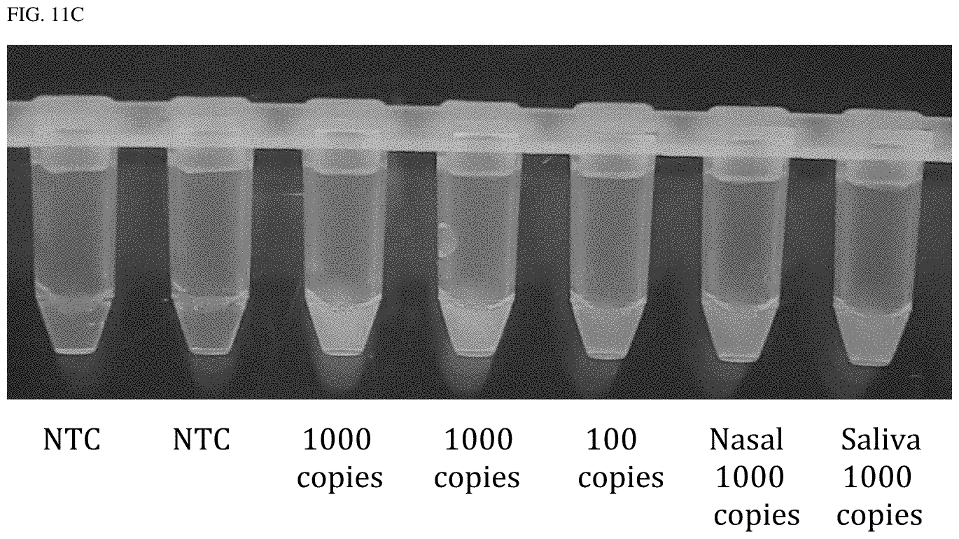

The presently preferred process to create lyophilized reagents begins by removing glycerol from the commercial enzymes as delivered by various suppliers (including New England Biolabs). For this, an ultrafiltration column with a 10 kDa cut-off limit was loaded with the DP-RT-LAMP enzymes. The enzyme storage buffer containing glycerol was exchanged with glycerol-free version enzyme storage buffer. Then, 10× primers mix and dNTPs were added. The mixture was lyophilized for 4 to 6 hours, leaving dry reagents as a white fluffy powder (FIG. 11A). Dry reagents were activated by rehydration buffer containing necessary salts and detergents and varying amount of viral RNA (1000 to 100 copies/assay). Alternatively, rehydration was done with contrived nasal and saliva samples (1000 copies of spiked RNA). Tt values were similar to their non-lyophilized versions (FIG. 11B) and clear visual fluorescent signals were recorded in all cases (FIG. 11C).

2. The Device that Reads the Generated Fluorescent Signals

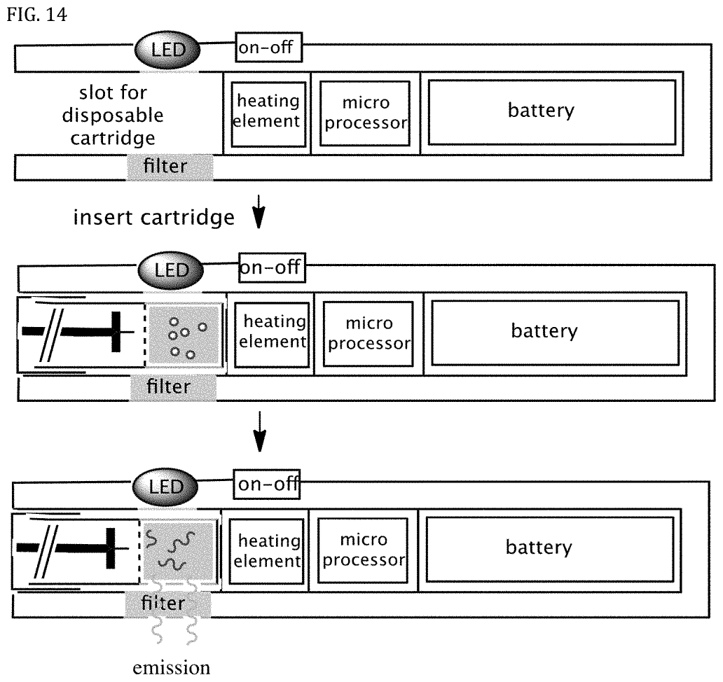

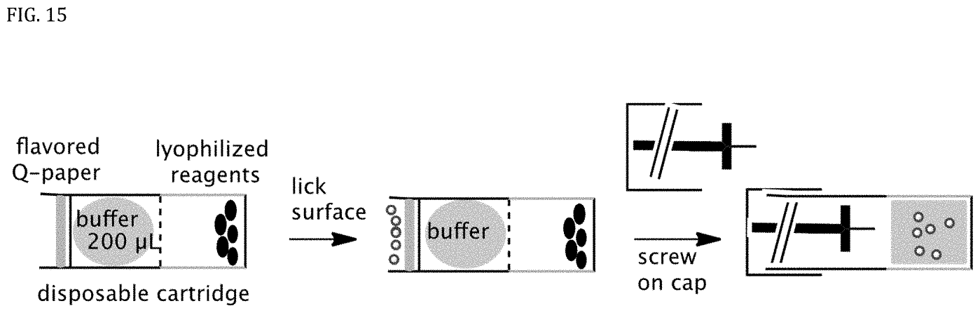

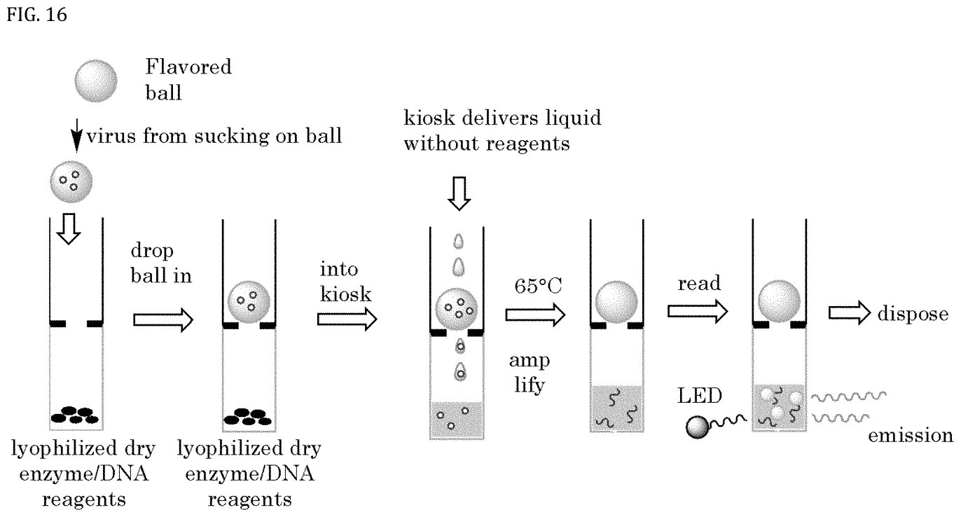

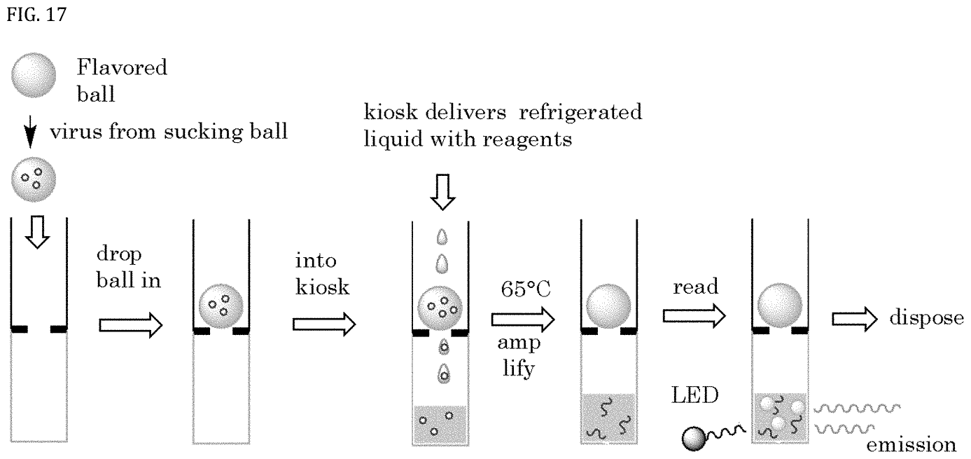

A schematic of the presently preferred implementation of the device invention is shown in FIG. 14. The personal coronavirus detector resembles an electronic cigarette. In the presently preferred implementation, it is battery powered. It contains a slot into which can be placed a disposable cartridge that, in its presently preferred implementation, performs a DP-RT-LAMP based on a sample of saliva. The disposable generates, in the presently preferred implementation, green fluorescence in 15-20 min if the saliva contains coronaviral RNA at a level sufficient that the user creates a forward infection risk for an environment that (s)he enters. In the presently preferred implementation, that fluorescence is observed via illumination with a 410 nm blue LED, and visual or cell phone capture of the fluorescence seen through an orange filter.

The utility of this device is apparent, as it will allow individuals to enter a dentist office, board an airplane, enroll for a semester at a university, or enter a workplace, with enhanced confidence that they will not contaminate fellow travelers, students, or workers. Its output may serve as an entry badge to public spaces

Also useful, the device may be used by individuals who have COVID who are self-quarantining. Through a cell phone app, they will deliver to epidemiologists who are working remotely daily reports of symptoms and viral loads. This will allow such individuals to constitute a distributed research lab to build a database of information about coronavirus biology.

### EXAMPLES

These examples illustrate several presently preferred embodiments of the instant invention. Alternatives may be substituted as is presently understood in the art.

### Example 1. Presently Preferred of the DP-RT-LAMP Primers and Displaceable Probes

TABLE 9
Presently preferred LAMP primers and probe sequences

| CoV2-W3 set | targeting S gene | |
|---|---|---|
| CoV2-W3-F3 | GAATCTCTCATCGATCTCC | SEQ ID NO 1 |
| CoV2-W3-FIP | AGCAAAGCATAATTGTCACCTTTTTGGCCATGGTACATTTGG | SEQ ID NO 2 |
| CoV2-W3-LF | GGCAATCAAGCCAGCTAT | SEQ ID NO 3 |
| CoV2-W3-LB | TGTGGATCCTGCTGCAA | SEQ ID NO 4 |
| CoV-2W3-BIP | GTCTCAAGGGCTGTTGTTCTTTTTGCTCAGAGTCGTCTTC | SEQ ID NO 5 |
| CoV2-W3-B3 | GACTCCTTTGAGCACTG | SEQ ID NO 6 |
| CoV2-W3-LB-tail12-5IBFQ | /5IABRFQ/GTGTCAAGAGCTCCGAGCCTCGTCGTTCATGCAATAGCGC- | SEQ ID NO 7 |
| | TGTGGATCCTGCTGCAA | |
| CoV2-tail12-40-Comp3FAM | GCGCTATTGCATGAACGACGAGGCTCGGAGCTCTTGACAC/36-FAM/ | SEQ ID NO 8 |
| CoV2-v2-4 set | targeting N gene | |
| CoV2-v2-4-F3 | ATGACGTTCGTGTTGTT | SEQ ID NO 9 |
| CoV2-v2-4-FIP | CTCCATTCTGGTTACTGCCATTTTTATCAGCGAAATGCACC | SEQ ID NO 10 |
| CoV2-v2-4-LF | TCTGAGGGTCCACCAAAC | SEQ ID NO 11 |
| CoV2-v2-4-LB | ATACTGCGTCTTGGTTCAC | SEQ ID NO 12 |
| CoV2-v2-4-BIP | CGGCCCCAAGGTTTACCTTTTTCCATGTTGAGTGAGAGC | SEQ ID NO 13 |
| CoV2-v2-4-B3 | GGTGTTAATTGGAACGC | SEQ ID NO 14 |
| CoV2-v2-4-LF-tail12-5IBFQ | /5IABRFQ/GTGTCAAGAGCTCCGAGCCTCGTCGTTCATGCAATAGCGC- | SEQ ID NO 15 |
| | TCTGAGGGTCCACCAAAC | |
| CoV2-tail12-40-Comp3FAM | GCGCTATTGCATGAACGACGAGGCTCGGAGCTCTTGACAC/36-FAM/ | SEQ ID NO 16 |
| RNaseP-2 set | targeting RNase P gene (internal control) | |
| RNaseP-2-F3 | GGAGAGTGAGTTGATCAG | SEQ ID NO 17 |
| RNaseP-2-FIP | ATAGCCCTCCTAGGCTCCTTTTTCCCTCTATCTGCAACTTG | SEQ ID NO 18 |
| RNaseP-2-LF | AGGCTTGCTTACCTCCAG | SEQ ID NO 19 |
| RNaseP-2-LB | CAGAGGCACCTAGGATTGG | SEQ ID NO 20 |
| RNaseP-2-BIP | TGGTGACCTGAACTAGGGTTTTTGTGCTGTGATCTGTCC | SEQ ID NO 21 |
| RNaseP-2-B3 | CTTTCCCTCATCCTTCTC | SEQ ID NO 22 |
| RNaseP-2-LB-tail13-5IBFQ | /5IABRFQ/GCAGCGGACGTCATAGGGACAATATCTTTCTCGCGCGGGA- | SEQ ID NO 23 |
| | CAGAGGCACCTAGGATTGG | |
| RNaseP-2-tail13-40- | TCCCGCGCGAGAAAGATATTGTCCCTATGACGTCCGCTGC/36-FAM/ | SEQ ID NO 24 |
| Comp3FAM | | |
| RNaseP-2-tail13-40- | TCCCGCGCGAGAAAGATATTGTCCCTATGACGTCCGCTGC/3Joe_N/ | SEQ ID NO 24 |
| Comp3JOE | | |

LAMP primers and strand displaceable probes were purchased from Integrated DNA Technologies (IDT, Coralville, Iowa) (Table 9). Strand-displaceable probes were 5′-quencher labeled with Iowa Black-FQ (IBFQ). Fluorescently labeled displaceable probes partially complementary to the quencher labeled probes were 3′-labeled with FAM. Alternatively, for multiplexing purposes, internal control probes targeting the human RNase P gene were 5′-labeled with IBFQ and 3′-labeled with JOE.

Underlined sequences are double strand segments of strand-displacing probes. FAM was used for SARS-CoV-2 detection and internal control targeting RNase P gene (λₑₓ-λₑₘ=495 nm-520 nm, color observed with excitation at 470 nm, green), JOE was used for RNase P gene for multiplexed LAMP experiments (λₑₓ-λₑₘ=529 nm-555 nm, color observed with excitation at 470 nm, yellow). Iowa Black FQ was used as a common quencher with absorption range of 420-620 nm. This quencher is typically used with fluorophores that emit in green to pink range of the visible spectrum. Presently preferred primers are listed in Table 9. Substantially identical primers are those that do not differ by more than two nucleotides within them, or by more than two nucleotides in length. The standard nucleotides in these primers may be replaced by their SAMRS equivalents.

### Example 2. Targets to Validate the Invention with SARS-CoV-2 RNA as the Target IVT RNA Fragment Preparation

Target RNA was generated from synthetic DNA fragments of the viral genes of interest. Synthetic DNA gene fragments were ordered from IDT as gBlocks. An initial PCR introduced the T7 promoter. Next, 150 nM of PCR product was used in T7 RNA transcription reaction (50 μL total volume); the reaction mixture was incubated at 37° C. for 16 h. DNA templates were removed by digestion with DNase I, the mixture was phenol-CHCl₃ extracted, and the RNA was recovered by EtOH precipitation. The product RNA was quantified using a Nanodrop UV spectroscopy, and reference materials with known concentrations were prepared in serial dilutions in TE buffer (10 mM Tris pH 7.0, 1 mM EDTA) and aliquots were stored at −80° C.

### Fully Synthetic SARS-CoV-2 RNA

Synthetic SARS-CoV-2 RNA Control was from Twist Bioscience (MT007544.1, 1×10⁶ RNA copies/μL). It was used for initial limit of detection (LOD) studies. Appropriate dilutions were made in 1 mM Na citrate pH 6.5, 0.4 U/μL RNase inhibitor (NEB, Ipswich, Mass.) and aliquots were stored at −80° C.

### Heat-Inactivated SARS-CoV-2 Isolate

Authentic SARS-CoV-2, isolate USA-WA 1/2020, was obtained through BEI Resources (cat no. NR-52286, 1.16×10⁹ genome equivalents/mL). This virus has been inactivated by heating at 65° C. for 30 minutes. Target dilutions were made in 1 mM Na citrate pH 6.5, 0.4 U/μL RNase inhibitor (NEB, Ipswich, Mass.) supplemented with 0.15 ng/μL human RNA and aliquots (100 μL) were stored at −80° C. This target was used to determine final LODs and spike-in experiments where minimal sample preparation methods were sought for nasal swab and saliva sampling.

### Example 3. The Presently Preferred DP-RT-LAMP Assay for 2019-nCoV

12.5 μL of 2× WarmStart LAMP master mix (NEB) was combined with 2.5 μL of 10× LAMP primer set, 1 μL of excess B3 primer (300 μM), 0.175 μL of dUTP (100 mM, Promega), 0.5 of Antarctic Thermolabile UDG (1 U/μL, NEB), 0.5 μL of RNase inhibitor (40 U/μL, NEB), 2 μL of template RNA or inactivated virus isolate, μL and 6 μL of nuclease-free water (or briefly processed nasal/saliva samples) to bring the final reaction volume to 25 μL.

10× LAMP primer set consists of 16 μM each of FIP and BIP, 2 μM each of F3 and B3, 5 μM LF (or LB for CoV2-v2-4 set), 4 μM LB (or LF for CoV2-v2-4 set), 150 nM quencher-bearing probe, and 100 nM of fluorophore-bearing probe.

Reactions were monitored in real-time using either a LightCycler® 480 (Roche Life Science, US) or a Genie® II (Optigene, UK) instrument. 8-strip PCR tubes were first incubated at 55° C. for 10 min followed by incubation at 65° C. for 45-60 min. During the 65° C. incubation, fluorescence signal was recorded every 30 seconds using FAM/SYBR channel of the instrument.

End-point observation of the fluorescence signal generated by strand displaceable probes was enabled by blue LED light (excitation at 470 nm) through orange filter of SafeBlue Illuminator/Electrophoresis System, MBE-150-PLUS (Major Science, US) or 3D printed observation box (Firebird Biomolecular Sciences, US).

### Example 4. The Preferred Process from Nasal Swabs

### Mid-Turbinate and Anterior Nasal Swab Sampling

CleanWIPE Swab, 3″ Semi-Flexible bulb tip (HT1802-500, Foamtec International) is used for nasal sampling. Each nostril is swabbed for at least 10 seconds using the same swab. The swab is placed in sterile 15 mL falcon tube and stored at 4° C. until processing.

The nasal swab was eluted in 100 μL of buffer solution (1 mM Na citrate pH 6.5, 2.5 mM TCEP, 1 mM EDTA, 10 mM LiCl, 15% Chelex-100) by brief vortexing. Swabs were then removed and elution solution was briefly spun down. 6 μL of sample elution was combined with 2 μL of inactivated virus (BEI resources) or water (no template control) and added to 17 μL of DP-RT-LAMP reaction mixture (12.5 μL of 2× WarmStart LAMP master mix, 2.5 μL of 10× LAMP primer set, 1 μL of excess B3 primer (300 μM), 0.175 μL of dUTP (100 mM), 0.5 μL of Antarctic Thermolabile UDG (1 U/μL, NEB), 0.5 μL of RNase inhibitor (40 U/μL, NEB). Samples were then incubated and analyzed in real-time as described above.

Other methods were tested. Thus, we initially tested TE buffer (10 mM Tris-HCl pH 7.0, 1 mM EDTA) for eluting nasal swabs. Another method involved use of an inactivation buffer containing 10 mM NaOH, 2.5 mM TCEP and 1 mM EDTA followed by incubation at 95° C. for 5 min.²⁷ Same buffer solution was then coupled with 15% Chelex-100 and extended heating (56° C. 15 min and 95° C. 5 min).

### Saliva to DP-RT-LAMP Testing

Saliva samples are collected before brushing the teeth or 1 hour after brushing the teeth. A sample of saliva is preferably −1 mL, collected in sterile 5 mL falcon tube and stored at 4° C. until processing and samples processed within 1 hour. Alternatively the saliva may be collected by having a saliva provider suck on a ball. This ball may optionally be semi-porous, or be flavored. 100 μL of 15% Chelex-100 in 1.6 mL microcentrifuge tube was spun down briefly and supernatant was removed. To that, 100 μL of saliva mixed with 1 μL of concentrated sample preparation solution (0.1 M Na citrate pH 6.5, 1M LiCl, 0.25 M TCEP, 0.1 M EDTA) was added. Each sample was briefly vortexed and spun down to settle Chelex-100 down. 6 μL of saliva sample was combined with 2 μL of inactivated virus (BEI resources) or water (no template control) and added to 17 μL of DP-RT-LAMP mixture and the reaction was done as previously described.

### Collecting Saliva on Q-Paper and DP-RT-LAMP Testing

Q-Paper Preparation

Whatman filter paper (1 gram) was immersed in 50 mL of 1.8% aq. NaOH solution for 10 min. Treated paper was collected by filtration and immersed in aq. EPTMAC (2,3-epoxypropyl) trimethylammonium chloride) solution for 24 h at RT. The mass ratio of EPTMAC to filter paper was 0.28 to 1. Cationic (Q) paper was collected by vacuum filtration and neutralized with 50 mL of 1% AcOH. Final product was washed three times with ethanol (96%) and dried at 55° C. for 1 h. Q-paper sheets were cut into small rectangles (˜0.5×0.2 cm) for saliva collection.

### Saliva on Q-Paper

Q-paper is first dipped into saliva samples and soaked for 5 seconds, then air dried for 5 min. Q-paper containing saliva is directly inserted into 50 μL of DP-RT-LAMP mixture (25 μL of 2× WarmStart LAMP master mix, 5 μL of 10× LAMP primer set, 2 μL of excess B3 primer (300 μM), 0.35 μL of dUTP (100 mM), 1 μL of Antarctic Thermolabile UDG (1 U/μL, NEB), 1 μL of RNase inhibitor (40 U/μL, NEB) and 16 μL of nuclease-free water) and reaction was proceeded as described above.

### Example 5. Biplexed DP-RT-LAMP to Detect SARS-CoV-2 and RNase P (Internal Control)

BEI 2019-nCoV Thermally Inactivated Virus in Human RNA Background

Varying amounts (10⁵, 10⁴, 10³, 10², 10 and 5 copies) of heat-inactivated SARS-CoV-2 (BEI resources) spiked with 440 copies of human RNA, 1.25 μL of 10× CoV2-W3 LAMP primer set (FAM-labeled probe) and 1.25 μL of 10× RNaseP-2 LAMP primer set (JOE-labeled probe) were added to DP-RT-LAMP mixture (25 μl total volume).

Multiplexed reaction mixtures were pre-incubated at 55° C. for 10 min, then 65° C. for 50 min and the fluorescence signals from three channels were recorded every 30 seconds using LightCycler® 480 (Roche Life Science, US) during 65° C. incubation. Channel 483-533 is specific to FAM-labeled SARS-CoV-2 probe, channel 523-568 is an intermediate channel for both FAM- and JOE-probes, and channel 558-610 is specific to JOE-labeled RNase P probe.

BEI 2019-nCoV Thermally Inactivated Virus Spiked into Nasal Swab Samples

6 μL of briefly processed nasal swab was combined with 2 μL of heat-inactivated SARS-CoV-2 isolate to give 10⁶ to 10³ RNA copies per assay. Nasal samples spiked with targets were added to DP-RT-LAMP mixture containing CoV2-W3 and RNAseP-2 LAMP primers in equal amounts (1.25 μL each of 10× LAMP primer sets) to give a total 25 μL assay volume. Real-time analysis was performed as mentioned above using LightCycler® 480 with three fluorescence channels.

### Example 6. Use of Lyophilized Reagents in DP-RT-LAMP

### Dialysis Example for 10 LAMP Reactions

10 μL of Bst 2.0 WarmStart® DNA Polymerase (8 U/μL, NEB), 10 μL of WarmStart® RTx Reverse Transcriptase (15 U/μL, NEB), 5 μL of Antarctic Thermolabile UDG (1 U/μL, NEB), 5 μL of RNase inhibitor (40 U/μL, NEB) was combined with 170 μL of dialysis buffer (10 mM Tris-HCl pH 7.5, 50 mM KCl, 1 mM DTT, 0.1 mM EDTA, 0.1% Triton X-100). 200 μL mixture was then placed in an ultrafiltration membrane (10 kDA cut-off limit, Millipore, Billerica, Mass.). Samples were centrifuged at 13,000 rpm for 8 min, then further washed with 250 μL of dialysis buffer twice to concentrate glycerol free enzyme mix down to 30 μL. 30 μL of enzyme mix was combined with 25 μL of 10× LAMP primer set, 10 μL of 300 μM B3 primer, 35 μL of dNTP mix (10 mM each of dATP, dCTP, dGTP and 5 mM each of dTTP and dUTP) and 25 μL of 1M D-(+)-trehalose. Combined mixture was then distributed into 8-strip PCR tubes as 12.5 μL aliquots. Samples were frozen by liquid nitrogen and lyophilized for 4-6 h. Lyophilized reagents were stored at RT and tested within 7 days.

### Reconstitution of Lyophilized LAMP Reagents

6 μL of sample (nasal swab/saliva or RNA template) is mixed 19 μL of reconstitution buffer (2.5 μL 10× isothermal amplification buffer (NEB), 1.5 μL 100 mM MgSO₄ and 15 μL of nuclease-free water) and added into lyophilized reagents. DP-RT-LAMP reactions are monitored in real-time using Genie II and fluorescence signal is visualized as described in previous sections.

What is claimed is:

1. A process to detect a segment of an RNA molecule presented in a biological sample, wherein said segment serves as a template for a displaceable probe reverse transcriptase loop amplification of said template in an amplification mixture, wherein
(a)(1) said template has six regions, in the following order from the 3′-end to the 5′-end, termed F3c, F2c, F1c, B1, B2, and B3,
(a)(2) said amplification mixture is provided an external primer, termed F3, that is substantially Watson-Crick complementary to F3c,
(a)(3) said amplification mixture is provided a first internal primer that has two regions, one F1c towards its 5′-end and the other F2 towards its 3′-end, where the two regions are joined by a linking oligonucleotide, and where F1c is substantially Watson-Crick complementary to F1 and F2 is substantially Watson-Crick complementary to F2c,
wherein polymerase-catalyzed extension of said first internal primer generates a first copy that comprises F1c, F2, F1, B1c, B2c, and B3c in the 5′- to 3′ direction, wherein F1c is substantially Watson-Crick complementary to F1, F2 is substantially Watson-Crick complementary to F2c, F1 is substantially Watson-Crick complementary to F1c, B1c is substantially Watson-Crick complementary to B1, B2c is substantially Watson-Crick complementary to B2, and B3c is substantially Watson-Crick complementary to B3, and then
(a)(4) said amplification mixture is provided a second external primer, termed B3, which is substantially Watson-Crick complementary to B3c and
(a)(5) said amplification mixture is provided a second internal primer that has two regions, one B1c towards its 5′-end and the other B2 towards its 3′-end, where the two regions are joined by a linking oligonucleotide, and where B1c is substantially Watson-Crick complementary to B1 and B2 is substantially Watson-Crick complementary to B2c,
wherein polymerase-catalyzed extension of said second internal primer generates a second copy that comprises B1c, B2, B1, F1c, F2c, and F1 in the 5′- to 3′ direction, said second copy can form a structure having two loops, and
(a)(6) said amplification mixture is provided a tagged primer that is a DNA molecule comprising two regions, the first tag region carrying a fluorescence quenching moiety at or near its 5′-end and the second tag region substantially Watson-Crick complementary to a region between B1 and B2 or a region between F1 and F2, and
(a)(7) said amplification mixture is provided a displaceable probe that is a DNA molecule having a fluorescent moiety at or near its 3′-end, said displaceable probe being substantially Watson-Crick complementary to the first tag region, wherein said amplification mixture contains a reverse transcriptase, and
(b) said biological sample is either as a nasal swab or a sample of saliva, with no sample preparation other than collection, transfer and dilution, and wherein detection comprises the observation of fluorescence arising from the displaceable probe either at the end of the amplification or during the amplification.

2. The process of claim 1, where said saliva sample is presented to said amplification mixture on a support having covalently attached quaternary ammonium salts.

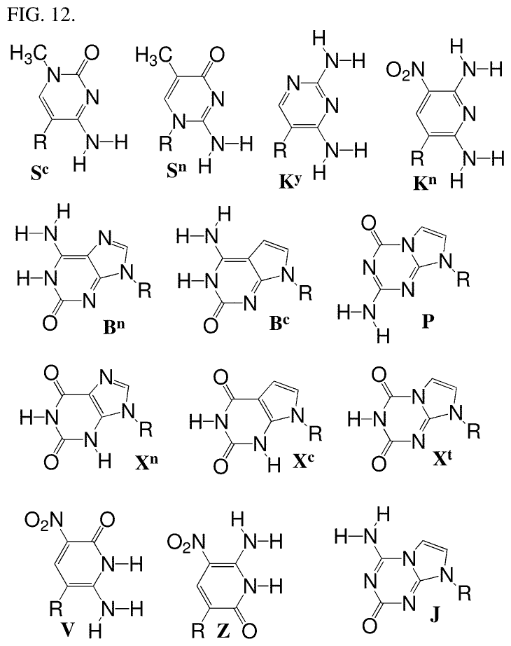

3. The process of claim 1, wherein one or more of the nucleotides within said tagged primer and probe are selected from the nucleotide analogs shown in FIG. 12.

4. The process of claim 1, wherein said primers comprise SEQ ID No 1 through SEQ ID NO 24.

5. The process of claim 1 wherein said primers comprise those substantially identical to SEQ ID No 1 through SEQ ID NO 24.

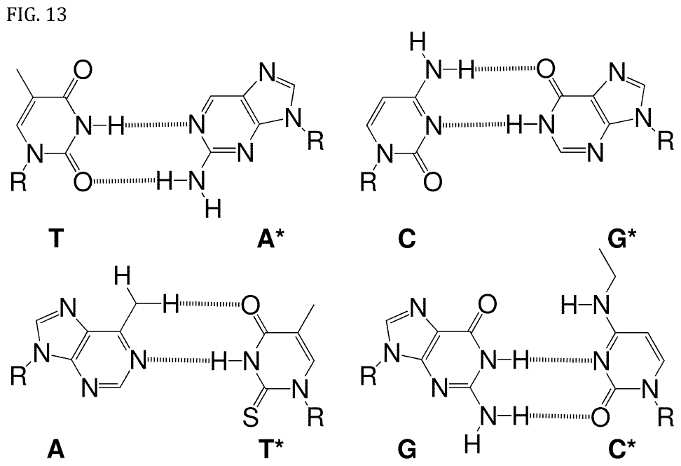

6. The process of claim 1, wherein one or more of the nucleotides within said tagged primer and probe are selected from the nucleotide analogs shown in FIG. 13.

7. The process of claim 1, wherein said incubation temperature is 60-70° C.

8. The process of claim 1, wherein time to detection is less than 25 minutes with 100 template molecules per sample.

9. The process of claim 1, wherein excess B3 is added.

10. The process of claim 1, wherein the mixture is pre-incubated at 55° C. prior to incubation at 60-70° C.

11. The process of claim 1, wherein nasal swab is subjected to an extraction with a solution that has a pH of 5 and 7.

12. The process of claim 1, wherein nasal swab is subjected to an extraction with a solution that contains Chelex-100 without any heat step.

13. The process of claim 1, wherein 100 copies of template are detected per assay in less than 18 min.

14. The process of claim 1, wherein the detection is done by direct observation of the fluorescence with the human eye.

15. The process of claim 1, wherein two primer sets may detect two different templates in a biplexed DP-RT-LAMP reaction.

16. The process of claim 1, wherein the amplification mixture comprises lyophilized reagents.

17. A handheld device to generate and detect a fluorescent signal arising if a segment of an RNA molecule is present in a sample, wherein said device comprises
(a) a slot wherein a disposable is placed,
(b) a microprocessor that controls a heater, wherein
(c) said heater warms a region of said disposable to a temperature between 50 and 70° C., wherein
(d) said disposable contains reagents generate a fluorescent signal when viral RNA is present,
(e) said device contains a light that illuminates the region of the disposable that generates the fluorescent signal, and
(f) a port allows the user to visualize the fluorescent signal,
wherein said fluorescent signal is generated by a process that involves the isothermal amplification of DNA molecules that contain segments substantially identical to, or substantially complementary to, said RNA molecule.

18. A kiosk that holds one or more slots that accepts a tube that contains a sample, and then delivers liquid to said tube and then warms a region of said tube to a temperature between 50 and 70° C., wherein tube and/or said liquid contains reagents generate a fluorescent signal when viral RNA is present in said sample, and wherein a light in said kiosk illuminates a region of said tube that generates the fluorescent signal, and said kiosk contains an element that detects the fluorescent signal.
wherein said fluorescent signal is generated by a process that involves the isothermal amplification of DNA molecules that contain segments substantially identical to, or substantially complementary to, said RNA molecule.

19. The kiosk of claim 18, wherein said sample is delivered on or near the surface of a ball that has been exposed to saliva.
